SEQUENCE LISTING (38 sequences)

SEQ ID NO: 1 - DNA, 246 bp, Human betaherpesvirus 5
  CMV enhancer nucleic acid sequence
ttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 2 - DNA, 278 bp, Artificial Sequence
  Description of Artificial Sequence Synthetic polynucleotide
  CBA promoter element nucleic acid sequence
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 3 - DNA, 40 bp, Artificial Sequence
  Description of Artificial Sequence Synthetic oligonucleotide
  Rabbit beta-globin splice acceptor nucleic acid sequence
ctctgctaaccatgttcatgccttcttctttttcctacag

SEQ ID NO: 4 - DNA, 315 bp, Artificial Sequence
  Description of Artificial Sequence Synthetic polynucleotide
  Spacer nucleic acid sequence
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagc

SEQ ID NO: 5 - DNA, 59 bp, Artificial Sequence
  Description of Artificial Sequence Synthetic oligonucleotide
  5′ spacer sequence
gcgttacataacttacggtaaatggcccgcctggctgaccgcccaacgacccccgccca

SEQ ID NO: 6 - DNA, 933 bp, Artificial Sequence
  Description of Artificial Sequence Synthetic polynucleotide
  CAG promoter
ttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 7 - protein, 187 aa, Homo sapiens
  Human IFNbeta protein
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 8 - DNA, 564 bp, Homo sapiens
  Human IFNbeta nucleic acid sequence
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaa

SEQ ID NO: 9 - DNA, 564 bp, Artificial Sequence
  Description of Artificial Sequence Synthetic polynucleotide
  Codon optimized human IFNbeta nucleic acid sequence
atgactaataagtgcctgctgcagatagccctgctcctgtgttttagcaccacggcactgagtatgtcctataatctgctgggctttctgcagaggtcatcaaacttccaatgccaaaagttgctctggcagctgaacggccggctggagtattgtctgaaagatcggatgaatttcgatattcccgaagaaatcaaacaattgcaacaatttcaaaaggaagatgcagccctgacgatttacgaaatgctgcaaaatattttcgcaatctttaggcaggacagcagctccacaggttggaacgaaaccatagtggaaaatctgctcgcgaacgtgtaccaccaaatcaatcacttgaaaaccgtgctcgaggagaagttggaaaaggaagactttacacgtggcaagcttatgtcttccttgcatctcaagaggtactacggccggatactccactatcttaaagctaaagaatattcccattgcgcgtggacaatcgtgcgcgttgagattctgcgcaatttctattttatcaatcgactgacgggctatttgcggaattga

SEQ ID NO: 10 - protein, 189 aa, Homo sapiens
  Human IFNalpha1 protein
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 11 - DNA, 570 bp, Homo sapiens
  Human IFNalpha1 nucleic acid sequence
atggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 12 - DNA, 570 bp, Artificial Sequence
  Description of Artificial Sequence Synthetic polynucleotide
  Codon optimized human IFNalpha1 nucleic acid sequence
atggcttctcctttcgcccttctcatggtactcgttgttttgtcctgtaaaagctcctgtagccttggatgcgaccttccggaaacacatagtcttgacaatcgaagaacactgatgttgcttgctcagatgtcacgcattagccccagcagttgccttatggatcgccacgatttcgggttcccgcaagaagaattcgacgggaatcaatttcagaaagcgcctgctatttcagtgctgcacgaattgattcaacaaatatttaatctgttcacaaccaaggacagttccgccgcctgggacgaagatcttctggataagttttgtacggagttgtatcaacaattgaacgatctggaagcttgcgtcatgcaagaagaacgcgtaggcgagaccccactcatgaacgcagattctattctggcagttaagaagtattttaggcggataacgctgtacttgactgaaaagaagtatagtccctgcgcttgggaagtggtgcgggccgagataatgcggtctcttagcctctctaccaatctgcaggagaggctgcgccgtaaagagtag

SEQ ID NO: 13 - protein, 166 aa, Homo sapiens
  Human IFNgamma protein
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 14 - DNA, 501 bp, Homo sapiens
  Human IFNgamma nucleic acid sequence
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 15 - DNA, 501 bp, Artificial Sequence
  Description of Artificial Sequence Synthetic polynucleotide
  Codon optimized human IFNgamma nucleic acid sequence
atgaagtacacctcttacatacttgccttccaactgtgtatagtcctgggaagcctggggtgctattgtcaagatccctacgtcaaggaagctgagaatctgaagaagtacttcaacgccggccactccgacgttgccgacaacggcacgctgtttctgggtatcctgaagaactggaaggaagaatctgatcgaaagatcatgcaatctcagatcgtgtctttctatttcaagctcttcaagaacttcaaggacgaccaatcaattcagaaaagcgtggaaacgataaaagaggatatgaacgttaaattctttaactccaacaagaagaagcgcgacgatttcgagaaattgacgaactacagcgttaccgatctcaacgtgcagagaaaggccatccacgagctgattcaggtaatggccgagctctcacctgcggccaagaccggcaaaagaaagcggagccaaatgcttttcaggggcagacgggcttcacaatga

SEQ ID NO: 16 - DNA, 53 bp, Artificial Sequence
  Description of Artificial Sequence Synthetic oligonucleotide
  Spacer sequence
ctcctgggcaacgtgctggttattgtgctgtctcatcattttggcaaagaatt

SEQ ID NO: 17 - protein, 19 aa, Teschovirus A
  P2A peptide
ATNFSLLKQAGDVEENPGP

SEQ ID NO: 18 - protein, 18 aa, Thosea asigna virus
  T2A peptide
EGRGSLLTCGDVEENPGP

SEQ ID NO: 19 - protein, 182 aa, Mus sp.
  Mouse IFNbeta protein
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 20 - DNA, 546 bp, Mus sp.
  Mouse IFNbeta nucleic acid sequence
aacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactaa

SEQ ID NO: 21 - protein, 186 aa, Canis lupus familiaris
  Canine IFNbeta protein
MTSRCILQTTLLLYFSTMALAMSNDLLRSQLSSSSLECQELLLQLNGTTEYCLKDRINFEIPEEIEKSRQFQKEDIILITHEMFQKIFDIFRRNISRTGWNETTVENLLVKLHWQKEHLEIILEDVKEKENFTWDNRTLLHLKKYYLRIVQYLKAKEYSICAWTIVQAEICRNFFFLNILTDYLQN

SEQ ID NO: 22 - DNA, 558 bp, Canis lupus familiaris
  Canine IFNbeta nucleic acid sequence
accagtagatgcatcctccaaacaactctcctgttgtatttctccaccatggctcttgccatgagcaacgacttgcttcgatcccagctaagcagcagcagtttggagtgtcaggagctcctattacagttgaatggaaccactgaatattgcctcaaggacaggataaacttcgagatccctgaggaaatcgagaaatcacgccagttccagaaggaggacatcatattgatcacccatgagatgttccagaagatctttgatattttcaggagaaatatctctagaacaggatggaatgagaccactgtcgagaaccttcttgtgaagctccactggcagaaggaacatctggagataatcctggaggacgtcaaagagaaggaaaacttcacctgggacaacaggactcttctgcacctgaagaaatattacttaaggattgtgcagtacctgaaggccaaggagtacagcatctgtgcctggacaatagtccaagcagaaatctgcaggaactttttcttccttaatatacttacagattatctccagaactga

SEQ ID NO: 23 - DNA, 589 bp, Artificial Sequence
  Description of Artificial Sequence Synthetic polynucleotide
  WPRE nucleic acid sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 24 - DNA, 122 bp, Macaca mulatta polyomavirus 1
  SV40 poly(A) nucleic acid sequence
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 25 - DNA, 2795 bp, Artificial Sequence
  Description of Artificial Sequence Synthetic polynucleotide
  AAV-CAG-hIFNb
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 26 - DNA, 2780 bp, Artificial Sequence
  Description of Artificial Sequence Synthetic polynucleotide
  AAV-CAG-mIFNb
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 27 - DNA, 2792 bp, Artificial Sequence
  Description of Artificial Sequence Synthetic polynucleotide
  AAV-CAG-cIFNb
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaccagtagatgcatcctccaaacaactctcctgttgtatttctccaccatggctcttgccatgagcaacgacttgcttcgatcccagctaagcagcagcagtttggagtgtcaggagctcctattacagttgaatggaaccactgaatattgcctcaaggacaggataaacttcgagatccctgaggaaatcgagaaatcacgccagttccagaaggaggacatcatattgatcacccatgagatgttccagaagatctttgatattttcaggagaaatatctctagaacaggatggaatgagaccactgtcgagaaccttcttgtgaagctccactggcagaaggaacatctggagataatcctggaggacgtcaaagagaaggaaaacttcacctgggacaacaggactcttctgcacctgaagaaatattacttaaggattgtgcagtacctgaaggccaaggagtacagcatctgtgcctggacaatagtccaagcagaaatctgcaggaactttttcttccttaatatacttacagattatctccagaactgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 28 - DNA, 2806 bp, Artificial Sequence
  Description of Artificial Sequence Synthetic polynucleotide
  AAV-CAG-hIFNa1
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 29 - DNA, 2737 bp, Artificial Sequence
  Description of Artificial Sequence Synthetic polynucleotide
  AAV-CAG-hIFNg
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 30 - DNA, 3343 bp, Artificial Sequence
  Description of Artificial Sequence Synthetic polynucleotide
  AAV-CAG-hIFNb-P2A-hIFNa1
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaaggaagcggcgccaccaatttcagcctgctgaaacaggccggcgacgtggaagagaaccctggccctcacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 31 - DNA, 3361 bp, Artificial Sequence
  Description of Artificial Sequence Synthetic polynucleotide
  AAV-CAG-hIFNb-P2A-hIFNg
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaaggaagcggcgccaccaatttcagcctgctgaaacaggccggcgacgtggaagagaaccctggccctatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 32 - DNA, 3907 bp, Artificial Sequence
  Description of Artificial Sequence Synthetic polynucleotide
  AAV-CAG-hIFNb-P2A-hIFNa1-T2A-hIFNg
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaaggaagcggcgccaccaatttcagcctgctgaaacaggccggcgacgtggaagagaaccctggccctcacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataaggaagcggcgagggcaggggaagtctactaacatgcggggacgtggaggaaaatcccggcccaatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 33 - DNA, 2800 bp, Artificial Sequence
  Description of Artificial Sequence Synthetic polynucleotide
  AAV-CAG-hIFNb-Codon Optimized
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgactaataagtgcctgctgcagatagccctgctcctgtgttttagcaccacggcactgagtatgtcctataatctgctgggctttctgcagaggtcatcaaacttccaatgccaaaagttgctctggcagctgaacggccggctggagtattgtctgaaagatcggatgaatttcgatattcccgaagaaatcaaacaattgcaacaatttcaaaaggaagatgcagccctgacgatttacgaaatgctgcaaaatattttcgcaatctttaggcaggacagcagctccacaggttggaacgaaaccatagtggaaaatctgctcgcgaacgtgtaccaccaaatcaatcacttgaaaaccgtgctcgaggagaagttggaaaaggaagactttacacgtggcaagcttatgtcttccttgcatctcaagaggtactacggccggatactccactatcttaaagctaaagaatattcccattgcgcgtggacaatcgtgcgcgttgagattctgcgcaatttctattttatcaatcgactgacgggctatttgcggaattgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 34 - DNA, 2806 bp, Artificial Sequence
  Description of Artificial Sequence Synthetic polynucleotide
  AAV-CAG-hIFNa1-Codon Optimized
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatggcttctcctttcgcccttctcatggtactcgttgttttgtcctgtaaaagctcctgtagccttggatgcgaccttccggaaacacatagtcttgacaatcgaagaacactgatgttgcttgctcagatgtcacgcattagccccagcagttgccttatggatcgccacgatttcgggttcccgcaagaagaattcgacgggaatcaatttcagaaagcgcctgctatttcagtgctgcacgaattgattcaacaaatatttaatctgttcacaaccaaggacagttccgccgcctgggacgaagatcttctggataagttttgtacggagttgtatcaacaattgaacgatctggaagcttgcgtcatgcaagaagaacgcgtaggcgagaccccactcatgaacgcagattctattctggcagttaagaagtattttaggcggataacgctgtacttgactgaaaagaagtatagtccctgcgcttgggaagtggtgcgggccgagataatgcggtctcttagcctctctaccaatctgcaggagaggctgcgccgtaaagagtaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 35 - DNA, 2737 bp, Artificial Sequence
  Description of Artificial Sequence Synthetic polynucleotide
  AAV-CAG-hIFNg Codon Optimized
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgaagtacacctcttacatacttgccttccaactgtgtatagtcctgggaagcctggggtgctattgtcaagatccctacgtcaaggaagctgagaatctgaagaagtacttcaacgccggccactccgacgttgccgacaacggcacgctgtttctgggtatcctgaagaactggaaggaagaatctgatcgaaagatcatgcaatctcagatcgtgtctttctatttcaagctcttcaagaacttcaaggacgaccaatcaattcagaaaagcgtggaaacgataaaagaggatatgaacgttaaattctttaactccaacaagaagaagcgcgacgatttcgagaaattgacgaactacagcgttaccgatctcaacgtgcagagaaaggccatccacgagctgattcaggtaatggccgagctctcacctgcggccaagaccggcaaaagaaagcggagccaaatgcttttcaggggcagacgggcttcacaatgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 36 - DNA, 3430 bp, Artificial Sequence
  Description of Artificial Sequence Synthetic polynucleotide
  AAV-CAG-hIFNb-P2A-hIFNa1 Codon Optimized
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgactaataagtgcctgctgcagatagccctgctcctgtgttttagcaccacggcactgagtatgtcctataatctgctgggctttctgcagaggtcatcaaacttccaatgccaaaagttgctctggcagctgaacggccggctggagtattgtctgaaagatcggatgaatttcgatattcccgaagaaatcaaacaattgcaacaatttcaaaaggaagatgcagccctgacgatttacgaaatgctgcaaaatattttcgcaatctttaggcaggacagcagctccacaggttggaacgaaaccatagtggaaaatctgctcgcgaacgtgtaccaccaaatcaatcacttgaaaaccgtgctcgaggagaagttggaaaaggaagactttacacgtggcaagcttatgtcttccttgcatctcaagaggtactacggccggatactccactatcttaaagctaaagaatattcccattgcgcgtggacaatcgtgcgcgttgagattctgcgcaatttctattttatcaatcgactgacgggctatttgcggaattgaggaagcggcgccaccaatttcagcctgctgaaacaggccggcgacgtggaagagaaccctggccctatggcttctcctttcgcccttctcatggtactcgttgttttgtcctgtaaaagctcctgtagccttggatgcgaccttccggaaacacatagtcttgacaatcgaagaacactgatgttgcttgctcagatgtcacgcattagccccagcagttgccttatggatcgccacgatttcgggttcccgcaagaagaattcgacgggaatcaatttcagaaagcgcctgctatttcagtgctgcacgaattgattcaacaaatatttaatctgttcacaaccaaggacagttccgccgcctgggacgaagatcttctggataagttttgtacggagttgtatcaacaattgaacgatctggaagcttgcgtcatgcaagaagaacgcgtaggcgagaccccactcatgaacgcagattctattctggcagttaagaagtattttaggcggataacgctgtacttgactgaaaagaagtatagtccctgcgcttgggaagtggtgcgggccgagataatgcggtctcttagcctctctaccaatctgcaggagaggctgcgccgtaaagagtaggatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 37 - DNA, 3361 bp, Artificial Sequence
  Description of Artificial Sequence Synthetic polynucleotide
  AAV-CAG-hIFNb-P2A-hIFNg Codon Optimized
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgactaataagtgcctgctgcagatagccctgctcctgtgttttagcaccacggcactgagtatgtcctataatctgctgggctttctgcagaggtcatcaaacttccaatgccaaaagttgctctggcagctgaacggccggctggagtattgtctgaaagatcggatgaatttcgatattcccgaagaaatcaaacaattgcaacaatttcaaaaggaagatgcagccctgacgatttacgaaatgctgcaaaatattttcgcaatctttaggcaggacagcagctccacaggttggaacgaaaccatagtggaaaatctgctcgcgaacgtgtaccaccaaatcaatcacttgaaaaccgtgctcgaggagaagttggaaaaggaagactttacacgtggcaagcttatgtcttccttgcatctcaagaggtactacggccggatactccactatcttaaagctaaagaatattcccattgcgcgtggacaatcgtgcgcgttgagattctgcgcaatttctattttatcaatcgactgacgggctatttgcggaattgaggaagcggcgccaccaatttcagcctgctgaaacaggccggcgacgtggaagagaaccctggccctatgaagtacacctcttacatacttgccttccaactgtgtatagtcctgggaagcctggggtgctattgtcaagatccctacgtcaaggaagctgagaatctgaagaagtacttcaacgccggccactccgacgttgccgacaacggcacgctgtttctgggtatcctgaagaactggaaggaagaatctgatcgaaagatcatgcaatctcagatcgtgtctttctatttcaagctcttcaagaacttcaaggacgaccaatcaattcagaaaagcgtggaaacgataaaagaggatatgaacgttaaattctttaactccaacaagaagaagcgcgacgatttcgagaaattgacgaactacagcgttaccgatctcaacgtgcagagaaaggccatccacgagctgattcaggtaatggccgagctctcacctgcggccaagaccggcaaaagaaagcggagccaaatgcttttcaggggcagacgggcttcacaatgagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

SEQ ID NO: 38 - DNA, 3994 bp, Artificial Sequence
  Description of Artificial Sequence Synthetic polynucleotide
  AAV-CAG-hIfNb-P2A-hIFNa1-T2A-hIFNg codon optimized
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatgactaataagtgcctgctgcagatagccctgctcctgtgttttagcaccacggcactgagtatgtcctataatctgctgggctttctgcagaggtcatcaaacttccaatgccaaaagttgctctggcagctgaacggccggctggagtattgtctgaaagatcggatgaatttcgatattcccgaagaaatcaaacaattgcaacaatttcaaaaggaagatgcagccctgacgatttacgaaatgctgcaaaatattttcgcaatctttaggcaggacagcagctccacaggttggaacgaaaccatagtggaaaatctgctcgcgaacgtgtaccaccaaatcaatcacttgaaaaccgtgctcgaggagaagttggaaaaggaagactttacacgtggcaagcttatgtcttccttgcatctcaagaggtactacggccggatactccactatcttaaagctaaagaatattcccattgcgcgtggacaatcgtgcgcgttgagattctgcgcaatttctattttatcaatcgactgacgggctatttgcggaattgaggaagcggcgccaccaatttcagcctgctgaaacaggccggcgacgtggaagagaaccctggccctatggcttctcctttcgcccttctcatggtactcgttgttttgtcctgtaaaagctcctgtagccttggatgcgaccttccggaaacacatagtcttgacaatcgaagaacactgatgttgcttgctcagatgtcacgcattagccccagcagttgccttatggatcgccacgatttcgggttcccgcaagaagaattcgacgggaatcaatttcagaaagcgcctgctatttcagtgctgcacgaattgattcaacaaatatttaatctgttcacaaccaaggacagttccgccgcctgggacgaagatcttctggataagttttgtacggagttgtatcaacaattgaacgatctggaagcttgcgtcatgcaagaagaacgcgtaggcgagaccccactcatgaacgcagattctattctggcagttaagaagtattttaggcggataacgctgtacttgactgaaaagaagtatagtccctgcgcttgggaagtggtgcgggccgagataatgcggtctcttagcctctctaccaatctgcaggagaggctgcgccgtaaagagtagggaagcggcgagggcaggggaagtctactaacatgcggggacgtggaggaaaatcccggcccaatgaagtacacctcttacatacttgccttccaactgtgtatagtcctgggaagcctggggtgctattgtcaagatccctacgtcaaggaagctgagaatctgaagaagtacttcaacgccggccactccgacgttgccgacaacggcacgctgtttctgggtatcctgaagaactggaaggaagaatctgatcgaaagatcatgcaatctcagatcgtgtctttctatttcaagctcttcaagaacttcaaggacgaccaatcaattcagaaaagcgtggaaacgataaaagaggatatgaacgttaaattctttaactccaacaagaagaagcgcgacgatttcgagaaattgacgaactacagcgttaccgatctcaacgtgcagagaaaggccatccacgagctgattcaggtaatggccgagctctcacctgcggccaagaccggcaaaagaaagcggagccaaatgcttttcaggggcagacgggcttcacaatgagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaa

(19) United States
(12) Patent Application Publication
(10) Pub. No.: US 2024/0207443 A1
(43) Pub. Date: Jun. 27, 2024

(54) GENE THERAPY FOR IMMUNO-ONCOLOGY APPLICATIONS

(71) Applicants: CHAN ZUCKERBERG BIOHUB, INC., SAN FRANCISCO, CA (US); THE REGENTS OF THE UNIVERSITY OF CALIFORNIA, Oakland, CA (US)

(72) Inventors: NICOLE K. PAULK, SAN FRANCISCO, CA (US); JOSEPH L DERISI, SAN FRANCISCO, CA (US); DAVID R. RALEIGH, SAN FRANCISCO, CA (US)

(73) Assignees: CHAN ZUCKERBERG BIOHUB, INC., SAN FRANCISCO, CA (US); THE REGENTS OF THE UNIVERSITY OF CALIFORNIA, OAKLAND, CA (US)

(21) Appl. No.: 18/012,164

(22) PCT Filed: Jun. 30, 2021
(86) PCT No.: PCT/US21/39983

§ 371 Date: May 25, 2023

Related U.S. Application Data
(60) Provisional application No. 63/046,211, filed on Jun. 30, 2020.

(51) Int. Cl. A61K 48/00; A61K 9/00; A61K 38/21; A61P 35/00; C12N 15/86

(52) U.S. Cl. CPC A61K 48/005; A61K 9/0019; A61K 9/0085; A61K 38/212; A61K 38/215; A61K 38/217; A61K 48/0075; A61P 35/00; C12N 15/86; C12N 2750/14143

(57) ABSTRACT

The invention provides compositions and methods for treatment of glioblastoma and other conditions. In particular, the invention provides a recombinant adeno-associated virus (AAV) vector comprising a transgene encoding one or more interferon polypeptides and a CAG promoter which directs the expression of the transgene. The compositions are particularly suitable for intratumoral administration in gene therapy applications.

### CROSS-REFERENCE TO RELATED APPLICATIONS

This application claims priority to U.S. provisional application No. 63/046,211, filed Jun. 30, 2020, the entire content of which is incorporated herein by reference.

### STATEMENT AS TO RIGHTS TO INVENTIONS MADE UNDER FEDERALLY SPONSORED RESEARCH AND DEVELOPMENT

This invention was made with Government support under Grant Numbers DK107607 and EB029374, awarded by the National Institutes of Health. The Government has certain rights in the invention.

### SEQUENCE LISTING SUBMITTED AS AN ASCII FORMAT FILE

This application contains a Sequence Listing that has been submitted electronically in ASCII format and is hereby incorporated by reference in its entirety. Said ASCII copy, created on Jun. 29, 2021, is named 103182-1247836-003010WO_SL.txt and is 82,034 bytes in size.

### BACKGROUND OF THE INVENTION

Glioblastoma (GBM) is the most common and deadliest primary malignant brain cancer, with 12,000 new diagnoses annually in the US and 225,000 deaths globally each year (GBD 2016 Brain and Other CNS Cancer Collaborators, “Global, regional, and national burden of brain and other CNS cancer, 1990-2016: a systematic analysis for the Global Burden of Disease Study 2016”. Lancet Neurol 18: 376-393 (2019)). A lack of effective therapies has led to a 5-year survival rate of 5% and an overall median survival of 14.6 months (Ostrom et al., “CBTRUS Statistical Report: Primary Brain and Other Central Nervous System Tumors Diagnosed in the United States in 2009-2013”. Neuro Oncol 18, v1-v75 (2016)). The current standard approach to treatment includes surgical resection followed by radiation and temozolomide (TMZ) chemotherapy (Stupp et al., “Radiotherapy plus concomitant and adjuvant temozolomide for glioblastoma”. N Engl J Med, 352: 987-996 (2005)). However, complete removal of the tumor has proven difficult, and GBM is often resistant to radiation and chemotherapy. Additionally, chemotherapy is challenging because of the blood brain barrier that prevents effective delivery of reagents.

Beyond tumor debulking surgery, and standard radiation therapy, therapies currently in use or in development include: small molecule chemotherapies, antibodies, medical devices, personalized CAR-T cell therapies, personalized vaccines, and oncolytic viral therapies. Each has limitations that influence efficacy, safety, or broad applicability for different GBM tumor types.

Type I interferons (IFNs) of the innate immune system play a vital pleiotropic role in helping to treat cancer, acting as endogenous host anti-tumor immuno-oncology agents (Zitvogel et al., “Type I interferons in anticancer immunity”, Nat Rev Immunol 15: 405-14 (2015)). Interferon alpha 1 (IFNα1 or IFNa1) and interferon beta (IFNβ or IFNb) do this through a variety of direct and indirect actions against tumor cells, including activating the JAK-STAT signaling pathway, recruiting T cells, activating NK cells, and acting as both pro-apoptotic agents and potent inhibitors of angiogenesis. Similarly, the type II IFN (interferon gamma, IFNγ or IFNg) has been implicated as an important effector molecule of anti-tumor immunity, capable of suppressing tumor growth through various mechanisms. However, clinical trials using IFNs to treat cancer have been hampered by secondary toxicity and the short half-life of IFNs in the circulation (Einhorn and Grander “Why do so many cancer patients fail to respond to interferon therapy?”, J Interferon Cytokine Res 16(4): 275-281 (1996)).

### BRIEF SUMMARY OF THE INVENTION

Methods and compositions for treating, preventing development of, slowing progression of, reversing, or ameliorating symptoms and signs of cancer are provided in this disclosure.

In one approach, the method disclosed herein comprises administering AAV vectors to a subject, where the administration results in expression of exogenously delivered interferon polypeptides to tumor cells in subjects in need of treatment. In some embodiments, the subject in need of treatment has cancer. In one aspect, the cancer is brain cancer, such as glioblastoma.

In one aspect disclosed herein is a method for treating a patient in need of treatment for glioblastoma, comprising administering a interferon (IFN) alpha, IFN beta, IFN gamma, a combination of any two of IFN-alpha, IFN-beta, and IFN gamma, or all three of IFN alpha, IFN beta and IFN gamma, wherein the administering comprises gene therapy with a single viral vector. In one approach, a combination of any two of IFN-alpha, IFN-beta, and IFN gamma, or all three of IFN alpha, IFN beta or IFN gamma are encoded in a polycistronic transgene and an AAV viral vector comprising the transgene is administered. In some embodiments interferon beta is administered. In some embodiments the viral vector is AAV and comprises adeno-associated virus 9 capsid. In an approach, the viral vector is administered by Convection Enhanced Delivery (CED).

In one approach, compositions of the invention in one form include a recombinant adeno-associated virus (rAAV or AAV) vector comprising an expression cassette comprising: (a) a CAG promoter, wherein the CAG promoter comprises (i) a first segment comprising a cytomegalovirus (CMV) enhancer sequence, (ii) a second segment comprising a chicken beta-actin (CBA) gene promoter element, (iii) a third segment comprising a spacer sequence, and (iv) a fourth segment comprising a rabbit beta-globin splice acceptor, wherein the order of the segments 5-prime to 3-prime is first, second, third, and fourth; and (b) a transgene comprising a sequence encoding a first interferon polypeptide, wherein the transgene is 3-prime to the CAG promoter, and wherein expression of the transgene is under the control of the CAG promoter.

In some aspects, the third segment is 250 nucleotides to 350 nucleotides in length measured from the 3-prime end of the CBA promoter and the 5′ end of the rabbit beta-globin splice acceptor.

In some aspects, (i) the first segment has the sequence of SEQ ID NO: 1, and/or (ii) the second segment has the sequence of SEQ ID NO: 2, and/or (iii) the third segment has the sequence of SEQ ID NO:4, and/or (iv) the fourth segment has the sequence of SEQ ID NO: 3.

In one aspect, the expression cassette does not comprise SEQ ID NO: 5.

In one aspect, the CAG promoter has the sequence of SEQ ID NO: 6.

In some aspects, the first interferon polypeptide is human interferon beta (hIFNβ). In one aspect, the sequence encoding the first interferon polypeptide is codon optimized for expression in human cells.

In some aspects, the transgene comprises a sequence encoding a second interferon polypeptide, wherein expression of the second interferon polypeptide is under control of the CAG promoter, wherein the sequence encoding the second interferon polypeptide is 3-prime from the sequence encoding the first interferon polypeptide, and wherein the second interferon polypeptide is human interferon alpha 1 (hIFNα1). In another aspect, the second interferon polypeptide is human interferon gamma (hIFNγ). In some aspects, the sequences encoding the first and second interferon polypeptides are codon optimized for expression in human cells.

In some aspects, the transgene comprises a sequence encoding a third interferon polypeptide, wherein expression of the third interferon polypeptide is under control of the CAG promoter, wherein the sequence encoding the third interferon polypeptide is 3-prime from the sequence encoding the second interferon polypeptide, and wherein the third interferon polypeptide is human interferon gamma. In one aspect, the sequences encoding the first, second and third interferon polypeptides are codon optimized for expression in human cells.

In some aspects, the first interferon polypeptide is hlFNα1 (also called “hlFNa1”). In one aspect, the sequence encoding the first interferon polypeptide is codon optimized for expression in human cells.

In some aspects, the first interferon polypeptide is hIFNγ. In one aspect, the sequence encoding the first interferon polypeptide is codon optimized for expression in human cells.

In some aspects, the first interferon polypeptide is mouse interferon beta (mIFNβ). In some aspects, the first interferon polypeptide is canine interferon beta (cIFNβ).

In some aspects, the sequence encoding the first interferon polypeptide and the sequence encoding the second interferon polypeptide are connected by a sequence encoding a first linker peptide and a sequence encoding a first self-cleaving peptide; and wherein the sequence encoding a first self-cleaving peptide is 3-prime from the sequence encoding a first linker peptide.

In some aspects, the sequence encoding the first interferon polypeptide and the sequence encoding the second interferon polypeptide are connected by a sequence encoding a first linker peptide and a sequence encoding a first self-cleaving peptide, and wherein the sequence encoding a first self-cleaving peptide is 3-prime from the sequence encoding a first linker peptide; and wherein the sequence encoding the second interferon polypeptide and the sequence encoding the third interferon polypeptide are connected by a sequence encoding a second linker peptide and a sequence encoding a second self-cleaving peptide, and wherein the sequence encoding a second self-cleaving peptide is 3-prime from the sequence encoding a second linker peptide.

In one aspect, the expression cassette further comprises a woodchuck hepatitis virus posttranscriptional regulatory element (WPRE). In one aspect, a transgene is located between the CAG promoter and the WPRE.

In one aspect, the expression cassette further comprises a polyadenylation signal.

In some aspects, the expression cassette comprises two adeno-associated virus (AAV) inverted terminal repeats (ITRs), wherein the CAG promoter and the transgene(s) are located between the two ITRs. In some aspects, the ITR is AAV1 ITR, AAV2 ITR, AAV3 ITR, AAV4 ITR, AAV5 ITR, AAV6 ITR, AAV7 ITR, AAV8 ITR, AAV-rh8 ITR, AAV9 ITR, AAV10 ITR, AAV-rh10 ITR, AAV11 ITR, or AAV12 ITR.

In one aspect, the expression cassette does not comprise an enhancer sequence other than the CMV enhancer sequence.

In some aspects, the invention provides an rAAV comprising a rAAV capsid and the rAAV vector. In some aspects, the rAAV capsid is AAV1, AAV2, AAV3, AAV3b, AAV4, AAV5, AAV6, AAV7, AAV8, AAV-rh8, AAV9, AAV9-hu14, AAV10, AAV-rh10, AAV11, AAV12, AAV-NP22, AAV-NP66, AAV-NP40, AAV-NP59, AAV-DJ, AAV-DJ/8, AAV-LK03, AAV-rh74, or AAV-hu37.

In some aspects, the invention provides an isolated cell comprising the rAAV vector or the rAAV.

In some aspects, the invention provides a pharmaceutical composition comprising the rAAV vector, the rAAV, or the isolated cell, and a pharmaceutically acceptable excipient.

The invention further includes a method for treating cancer in a mammal in need of treatment comprising administering the rAAV vector, the rAAV, the isolated cell, or the pharmaceutical composition. In one aspect, the cancer is glioblastoma. In some aspects, the subject is a human, a mouse, or a dog. In one approach, the method comprises administering the rAAV vector, and the rAAV vector is administered by Convection Enhanced Delivery (CED). In one approach, the rAAV vector is administered by intratumoral injection (also called “intratumoral injection”) using CED. In some approaches, the rAAV vector is administered by intracranial injection, intracerebral injection, intracerebroventricular, or injection into the cerebrospinal fluid (CSF) via the cerebral ventricular system, cisterna magna, or intrathecal space.

In one aspect, invention provides the use of the rAAV vector, the rAAV, the isolated cell, or the pharmaceutical composition for the preparation of a medicament for treating cancer. In another aspect, the rAAV vector, the rAAV, the isolated cell, or the pharmaceutical composition are used for the preparation of a medicament for treating cancer.

### BRIEF DESCRIPTION OF THE DRAWINGS

FIG. 1A shows bioluminescence (BLI) signal measured in wild type mice transplanted with mouse GL261-FLuc cells after treatment with AAV9-mIFNβ, AAV9-GFP or PBS. FIG. 1B shows the overall survival of the mice transplanted with mouse GL261-FLuc cells after treatment with AAV9-mIFNβ, AAV9-GFP or PBS.

FIG. 2A shows bioluminescence (BLI) signal measured in athymic mice transplanted with human GBM6-Fluc cells after treatment with AAV9-hIFNβ, AAV9-GFP or PBS. FIG. 2B shows the overall survival of the human xenograft mice after treatment with AAV9-hIFNβ, AAV9-GFP or PBS, and harvested at an early time point. FIG. 2C shows BLI signal measured in athymic mice transplanted with human GBM6-Fluc cells after treatment with AAV9-GFP, or AAV9-hIFNβ at either a high (1.89e12vg) or low dose (1.89e12vg). FIG. 2D shows the overall survival of the human xenograft mice after treatment with AAV9-GFP or AAV9-hIFNβ (at two doses), and harvested at a late time point.

FIG. 3 shows the overall survival of athymic mice transplanted with human patient-derived tumor cells after treatment with AAV9-hIFNβ, AAV9-GFP or PBS, and harvested at an early time point.

FIG. 4 shows overall survival of mice transplanted with canine patient derived tumor cells after treatment with AAV9-hIFNβ (high or low dose), AAV9-GFP or PBS.

FIG. 5A shows tumor growth rates for the AAV9-hIFNβ treated condition and the control group. FIG. 5B shows fold changes in area at week 1 and week 2 for the AAV9-hIFNβ treated condition and the control condition.

FIG. 6A shows bioluminescent signal at week 1, 2 and 3 for human glioblastoma spheroids in the control condition. FIG. 6B shows bioluminescent signal at week 1, 2 and 3 for human glioblastoma spheroids in the AAV9-hIFNβ treated condition.

FIG. 7 quantifies the bioluminescent signal of adherent cultures at week 1, 2 and 3.

FIG. 8 shows expression of vectorized engineered interferon cytokines exhibit potent and selective tumor killing in human glioblastoma organoids. Quantified fluorescent glioblastoma tumor intensity by day following treatment. Mean and 95% confidence intervals (shaded area) are shown in intensity plots for the 6 replicate wells for each condition and time.

FIG. 9A-D shows significant mOS improvement and complete durable responses in a human glioblastoma xenograft model. FIG. 9A Quantified live bioluminescent (BLI) imaging of tumor growth in FLuc+ human glioblastoma tumors. Mean tumor size curves for saline treated mice (CED on day 9 post-transplantation) are shown by heavy solid line; AAV9-GFP treated mice by light solid line; AAV9-hIFNβ shown by dashed line. P<0.03 by unpaired T-test on day 22. n=30 (10 per treatment cohort). FIG. 9B. Kaplan Meyer survival curves. P<0.001 by log-rank (Mantel-Cox) test. CR=complete response. FIG. 9C Quantified live BLI imaging of tumor growth in FLuc+human glioblastoma tumors. Mean tumor size curves for AAV9-GFP treated mice (CED on day 9 post-transplantation) are shown by light solid line; AAV9-hIFNβ high dose shown in dashed line. P<0.02 by unpaired T-test on day 22 and P<0.0004 by unpaired T-test on day 35. n=30 (10 per treatment cohort). FIG. 9D Kaplan Meyer survival curves from dose response study. P<0.001-0.0002 by log-rank (Mantel-Cox) test.

FIG. 10A-C shows that treated allograft and PDX mice demonstrate the safety and efficacy of vectorized IFNβ expression against glioblastoma in orthogonal disease models. FIG. 10A. Quantified live BLI imaging of tumor growth in FLuc+ mouse glioblastoma tumors. Mean tumor size curves for saline treated mice (CED on day 5 post-transplantation) are shown by a light solid line; AAV9-GFP treated mice in heavy solid line; AAV9-mIFNB shown by dashed line. n=29. FIG. 10B. Kaplan Meyer survival curves for treated allograft mice. FIG. 10C. Kaplan Meyer survival curves for treated PDX mice. P<0.04 by log-rank (Mantel-Cox) test. CR=complete response.

FIG. 11 shows comparative anti-tumor effects of codon-optimized innate interferon cytokines on human glioblastoma organoids. Quantified total glioblastoma tumor intensity by day following treatment. Mean and 95% confidence intervals (shaded area) are shown in intensity plots for the 6 replicate wells for each condition and time.

### DETAILED DESCRIPTION OF THE INVENTION

### 1. Introduction

Interferon (IFN) polypeptides such as such as IFNβ, IFNα, and IFNγ have tumor suppressing properties. For example, IFNs have cell intrinsic properties useful against tumors (e.g. pro-apoptotic, cytostatic, drug sensitizing effects), and cell extrinsic properties (i.e. recruitment of immune cells like T cells and NK cells to kill the tumor). The inventors have developed methods and reagents for treatment of brain cancer and other cancers, inter alia, by delivering interferon(s) (IFN(s)) to tumor cells. As described herein, a series of AAV vectors expressing one or more interferon proteins have been developed to inhibit tumor growth, and the inventors have demonstrated and increase overall survival in numerous orthotopic mouse models of glioblastoma. For example, in a human xenograft mouse model of glioblastoma, mice treated with AAV9-hIFNβ survived more than 2-fold longer than control mice that had not received the treatment (see, e.g., Example 3, below). Thus, in one aspect hIFNβ-encoding AAV vectors of the invention, extend survival when administered to human xenograft mouse model of glioblastoma, relative to control animals not receiving hIFNβ. In some embodiments the average time of survival of treated mice is at least twice that of control mice administered PBS. In some embodiments the average time of survival of treated mice is 50% greater (1.5-fold) or 25% greater (1.25-fold) that of control mice.

In some approaches the rAAV vector contains a transgene that encodes a single interferon polypeptide. In some approaches the rAAV vector contains a bicistronic or tricistronic transgene that encodes multiple interferon polypeptides.

### Vector, Transgene and Expression Cassette

As used herein, “vector” may refer to a virus (e.g., an infectious viral particle comprising a transgene-containing expression cassette and structural capsid proteins derived from an adeno-associated virus capsid serotype) or may refer to the genetic cargo delivered by the virus, as will be apparent from context. The virus may be an adeno-associated virus (AAV or rAAV) such as AAV serotype 9. The transgene may include AAV ITRs. As used herein, “transgene” refers to the entire genetic cargo delivered by the virus to a cell including protein coding sequences and regulatory sequences. “Transgene,” “cargo” and “expression cassette” are used interchangeably. As used herein, “rAAV” and “AAV” are used interchangeably.

Aspects of the disclosure relate to rAAV vectors comprising an expression cassette. The expression cassette may comprise a transgene that encodes protein(s) to be delivered to a cell or tissue, as well as regulatory elements controlling expression of encoded protein(s). Regulatory elements include promoters, enhancers, terminator sequences, polyadenylation sequences, and the like), mRNA stability sequences (e.g. Woodchuck Hepatitis Virus Posttranscriptional Regulatory Element; WPRE), sequences that allow for internal ribosome entry sites (IRES) of bicistronic mRNA, sequences necessary for episome maintenance (e.g., ITRs), sequences that avoid or inhibit viral recognition by Toll-like or RIG-like receptors (e.g. TLR-7, -8, -9, MDA-5, RIG-I and/or DAI) and/or sequences necessary for transduction into cells.

In one approach the expression cassette includes a CAG promoter operably linked to a transgene encoding one or more interferon polypeptides. In some embodiments, the CAG promoter comprises a first segment comprising a cytomegalovirus (CMV) enhancer sequence, a second segment comprising a chicken beta-actin (CBA) gene promoter element, a third segment comprising a spacer sequence, and a fourth segment comprising a rabbit beta-globin splice acceptor. In some aspects, the order of the segments 5-prime to 3-prime is first, second, third, and fourth. In some embodiments the CAG promoter has a sequence of SEQ ID NO:6.

### Gene Therapy

In some approaches, gene therapy involves delivering interferon polypeptides into cells of a mammalian subject using rAAV vectors described herein. In one embodiment, the method disclosed herein comprises administering the rAAV vectors to a subject, where the administration results in expression of exogenously delivered interferon polypeptides to tumor cells in subjects in need of treatment. In some embodiments, the subject in need of treatment has cancer. In one aspect, the cancer is brain cancer, such as glioblastoma.

### 2. Recombinant Adeno-Associated Virus (Raav) Expression Cassettes

In one aspect, the present disclosure provides a recombinant adeno-associated virus (rAAV) vector comprising an expression cassette comprising (a) a CAG promoter and (b) a transgene comprising a sequence encoding a first interferon polypeptide. The transgene is 3-prime to the CAG promoter and expression of the transgene is under the control of the CAG promoter.

### CAG Promoter

As used herein, the term “CAG promoter” refers to a regulatory construct comprising, in a 5′ to 3′ sequence, a cytomegalovirus (CMV) enhancer, a chicken beta-actin (CBA) gene promoter element, a spacer, and a rabbit beta-globin splice acceptor.

### CMV Enhancer

The CMV enhancer, derived from the human CMV, contains various repeated sequence elements, and has been described in Boshart et al. “A very strong enhancer is located upstream of an immediate early gene of human cytomegalovirus”, Cell 41(2): 521-530 (1985), and in U.S. Pat. Nos. 5,168,062 and 5,385,839, each of which is incorporated herein by reference. In some approaches, the CMV enhancer has the nucleic acid sequence of SEQ ID NO:1. It will be understood that some variation in sequence is tolerated with little or no diminution of enhancer activity and, in some embodiments, the CMV enhancer used in the present invention differs from SEQ ID NO: 1 at one or more bases. In some approaches, the nucleic acid sequence of the CMV enhancer shares significant sequence identity, e.g., at least 90%, at least 91%, at least 92%, at least 93%, at least 94%, at least 95%, at least 96%, at least 97%, at least 98%, or at least 99% nucleic acid sequence identity with SEQ ID NO: 1.

In some approaches, the expression cassette does not comprise the nucleic acid sequence of SEQ ID NO: 5. In some approaches, the expression cassette does not comprise an enhancer sequence other than the CMV enhancer sequence.

### CBA Promoter Element

In some approaches, the CAG promoter comprises a chicken beta-actin (CBA) gene promoter element. In some approaches, the CBA gene promoter element comprises a CBA gene promoter sequence, a CBA gene first exon, and a CBA gene first intron. In some approaches, the CBA gene promoter element has the nucleic acid sequence of SEQ ID NO: 2. In some approaches, the nucleic acid sequence of the CBA gene promoter element shares significant sequence identity, e.g., at least 90%, at least 91%, at least 92%, at least 93%, at least 94%, at least 95%, at least 96%, at least 97%, at least 98%, or at least 99% nucleic acid sequence identity with SEQ ID NO: 2.

### Spacer Sequence

In some approaches, the CAG promoter comprises a spacer sequence immediately 3′prime to the CBA promoter element. In some approaches, the spacer sequence is at least 250 nucleotides in length. In some embodiments the spacer sequence is 250 to 350 nucleotides in length. In some approaches, the spacer sequence has the nucleic acid sequence of SEQ ID NO: 4. In some approaches, the nucleic acid sequence of the spacer sequence shares significant sequence identity, e.g., at least 90%, at least 91%, at least 92%, at least 93%, at least 94%, at least 95%, at least 96%, at least 97%, at least 98%, or at least 99% nucleic acid sequence identity with SEQ ID NO: 4.

### Splice Acceptor

In some approaches, the CAG promoter comprises a rabbit beta-globin splice acceptor. In some approaches, the rabbit beta-globin splice acceptor has the nucleic acid sequence of SEQ ID NO: 3. In some approaches, the nucleic acid sequence of the rabbit beta-globin splice acceptor shares significant sequence identity, e.g., at least 90%, at least 91%, at least 92%, at least 93%, at least 94%, at least 95%, at least 96%, at least 97%, at least 98%, or at least 99% nucleic acid sequence identity with SEQ ID NO: 3.

### Promoter

In some approaches, the CAG promoter has the nucleic acid sequence of SEQ ID NO: 6.

### 2.1 Transgenes Encoding Interferon Polypeptide(S)

In some approaches, the expression cassette comprises a transgene comprising a sequence encoding a first interferon polypeptide. The transgene is 3-prime to the CAG promoter and expression of the transgene is under the control of the CAG promoter.

Sequence identifiers for Interferon proteins are provided in Table 1, below, and in Section 9. It will be understood that certain modifications may be made to the amino acid sequence of an IFN protein without loss of biological function. For example, a transgene may have polynucleotide sequence that encodes an IFN variant that differs from a native sequence. In one embodiment the variant differs from the native sequence at 1, 2, 3, 4, 5, 6, 7, 8, 9, 10 or 1-20 residues.

TABLE 1

| SEQ ID NO: | IFN |
|---|---|
| 7 | Human IFNβ protein |
| 10 | Human IFNα1 protein |
| 13 | Human IFNγ protein |
| 21 | Canine IFNβ protein |

In some embodiments, the first interferon polypeptide is human interferon beta (hIFNβ). In other embodiments, the first interferon polypeptide is human interferon alpha 1 (hlFNα1). In some embodiments, the first interferon polypeptide is human interferon gamma (hIFNγ). In some embodiments the first interferon polypeptide is murine interferon beta (mIFNβ). In some embodiments the first interferon polypeptide is canine interferon beta (cIFNβ). TABLE 1 provides examples of transgene organization for various single IFN polypeptides, along with exemplary sequences. See Section 10, below. In TABLE 2, sequences 33, 34 and 35 comprise sequences that are human codon optimized. See Section 2.2, below.

TABLE 2

| | | Exemplary |
|---|---|---|
| Construct | Organization in AAV | Sequence |
| hIFNβ | CAG-hIFNb-WPRE-SV40pA | 25, 33 |
| hIFNα1 | CAG-hIFNα1-WPRE-SV40pA | 28, 34 |
| hIFNγ | CAG-hIFNg-WPRE-SV40pA | 29, 35 |
| mIFNβ | CAG-mIFNb-WPRE-SV40pA | 26 |
| cIFNβ | CAG-cIFNb-WPRE-SV40pA | 27 |

### Bi- and Tri-Cistronic Transgenes

In some versions a single AAV vector encodes multiple different IFN coding sequences, e.g., more than one interferon selected from human IFNβ, IFNα1 and IFNγ. Methods are known in the art for making multicistronic vectors for coordinated expression of multiple proteins, including, for illustration, internal ribosome entry site (IRES), ribosome skipping element (RSE), and self-cleaving peptide sites (e.g., furin cleavage sites). See, e.g., Shaimardanova et al., “Production and Application of Multicistronic Constructs for Various Human Disease Therapies” Pharmaceutics 11:580, 2019) and Section 2.3, below.

In some aspects of the invention, the transgene comprises a sequence encoding an additional, second interferon polypeptide. The sequence encoding the second interferon polypeptide is 3-prime from the sequence encoding the first interferon polypeptide. In some embodiments, expression of the second interferon polypeptide is under control of the CAG promoter.

In some embodiments, the first interferon polypeptide is hIFNβ and the second interferon polypeptide is hIFNα1. In some embodiments, the first interferon polypeptide is hIFNβ and the second interferon polypeptide is hIFNγ. TABLE 2 provides examples of transgene organization for various bicistronic constructs for delivering IFN polypeptides, along with exemplary sequences. See Section 10, below. In TABLE 3, sequences 36 and 37 comprise sequences that are human codon optimized. See Section 2.2, below.

TABLE 3

| | | Exemplary |
|---|---|---|
| Construct | Organization in AAV | Sequence |
| hIFNβ-hIFNα1 | CAG-hIFNb-GSG-P2A-hIFNa-WPRE- | 30, 36 |
| | SV40pA | |
| hIFNβ-hIFNγ | CAG-hIFNb-GSG-P2A-hIFNg-WPRE- | 31, 37 |
| | SV40pA | |

In some embodiments, the transgene comprises a sequence encoding a second interferon polypeptide and a third interferon polypeptide. In some embodiments, expression of the third interferon polypeptide is under control of the CAG promoter, and the sequence encoding the third interferon polypeptide is 3-prime from the sequence encoding the second interferon polypeptide.

In some embodiments, the first interferon polypeptide is hIFNβ, the second interferon polypeptide is hlFNα1, and the third interferon polypeptide is hIFNγ. TABLE 4 provides an example of transgene organization for an exemplary tri-cistronic construct for delivering hIFNβ, hlFNα1, and hIFNγ, along with exemplary sequences. See Section 10, below. In TABLE 3, sequence 38 comprise sequences that are human codon optimized. See Section 2.2, below.

TABLE 4

| | | Exemplary |
|---|---|---|
| Construct | Organization in AAV | Sequence |
| hIFNβ-hIFNα1- | CAG-hIFNb-GSG-P2A-hIFNa-GSG- | 32, 38 |
| hIFNγ | T2A-hIFNg-WPRE-SV40pA | |

It will be appreciated that the transgene constructs listed in Tables 1, 2 and 3, above are examples and that the invention is not limited to these particular constructs. For example, the invention comprises vector genomes with any of the following organizations: IFNβ, hlFNα1 and hIFNγ may comprise any transgenes with, or illustration and not limitation, any one of the following positions: hIFNβ; hlFNα1; hIFNγ; hIFNβ-hIFNα1; hIFNβ-hIFNγ; hIFNα1-hIFNβ; hIFNγ-hIFNβ; hIFNβ-hIFNα1-hIFNγ; hIFNβ-hIFNγ-hlFNα1; and hIFNγ-hIFNβ-hIFNγα1.

### 2.2 Codon Optimization

In some embodiments, transgene sequences are codon optimized for expression of an interferon or other polypeptide protein in a species or cell type of interest. Codon optimization can be used to increase the rate of translation or to produce recombinant RNA transcripts having desirable properties, such greater expression efficiency, as compared with transcripts produced using a non-optimized sequence. In particular embodiments, the nucleic acid sequence encoding the first, second, or the third interferon polypeptide is codon optimized for expression in human cells. Methods for codon optimization are readily available, for example, optimizer, accessible free of charge at http://genomes.urv.es/OPTIMIZER, and GeneGPS® Expression Optimization Technology from DNA 2.0 (Newark, California). See Raab et al., “The GeneOptimizer Algorithm: using a sliding window approach to cope with the vast sequence space in multiparameter DNA sequence optimization” Syst Synth Biol 4: 215 (2010).

### Human Interferon Beta

In some embodiments, the human interferon beta (hIFNβ) has the sequence represented by UniProt/SwissProt Database Entry No. P01574 (SEQ ID NO: 7). In some approaches, the hIFNβ is encoded by the nucleic acid sequence of SEQ ID NO: 8. In some approaches, the nucleic acid sequence encoding hIFNβ shares at least 90%, at least 91%, at least 92%, at least 93%, at least 94%, at least 95%, at least 96%, at least 97%, at least 98%, or at least 99% nucleic acid sequence identity with the nucleic acid sequence of SEQ ID NO: 8.

Codon Optimized hIFNβ

In one aspect, the nucleic acid sequence encoding hIFNβ is codon optimized for expression in human cells and has the nucleic acid sequence of SEQ ID NO: 9.

### Human Interferon Alpha 1

In some embodiments, the human interferon alpha 1 (hIFNα1) has the sequence represented by UniProt/SwissProt Database Entry No. P01562 (SEQ ID NO: 10). In some approaches, the hIFNα1 is encoded by the nucleic acid sequence of SEQ ID NO: 11. In some approaches, the nucleic acid sequence encoding hlFNα1 shares at least 90%, at least 91%, at least 92%, at least 93%, at least 94%, at least 95%, at least 96%, at least 97%, at least 98%, or at least 99% nucleic acid sequence identity with the nucleic acid sequence of SEQ ID NO: 11.

Codon Optimized hlFNα1

In one aspect, the nucleic acid sequence encoding hlFNα1 is codon optimized for expression in human cells and has the nucleic acid sequence of SEQ ID NO: 12.

### Human Interferon Gamma

In some embodiments, the human interferon gamma (hIFNγ) has the sequence represented by UniProt/SwissProt Database Entry No. P01579 (SEQ ID NO: 13). In some approaches, the hIFNγ is encoded by the nucleic acid sequence of SEQ ID NO: 14. In some approaches, the nucleic acid sequence encoding hIFNγ shares at least 90%, at least 91%, at least 92%, at least 93%, at least 94%, at least 95%, at least 96%, at least 97%, at least 98%, or at least 99% nucleic acid sequence identity with the nucleic acid sequence of SEQ ID NO: 14.

Codon Optimized hIFNγ

In one aspect, the nucleic acid sequence encoding hIFNγ is codon optimized for expression in human cells and has the nucleic acid sequence of SEQ ID NO: 15.

### Murine Interferon Beta

In some embodiments, the first interferon polypeptide is mouse interferon beta (mIFNβ). In some embodiments, the mouse interferon beta (mIFNβ) has the sequence represented by UniProt/SwissProt Database Entry No. P01575 (SEQ ID NO: 19). In some approaches, the mIFNB is encoded by the nucleic acid sequence of SEQ ID NO: 20. In some approaches, the nucleic acid sequence encoding mIFNβ shares at least 90%, at least 91%, at least 92%, at least 93%, at least 94%, at least 95%, at least 96%, at least 97%, at least 98%, or at least 99% nucleic acid sequence identity with the nucleic acid sequence of SEQ ID NO: 20.

### Canine Interferon Beta

In some embodiments, the first interferon polypeptide is canine interferon beta (cIFNβ). In some embodiments, the canine interferon beta (cIFNβ) has the sequence represented by UniProt/UniProtKB Database Entry No. B6E116 (SEQ ID NO: 21). In some approaches, the cIFNB is encoded by the nucleic acid sequence of SEQ ID NO: 22. In some approaches, the nucleic acid sequence encoding cIFNβ shares at least 90%, at least 91%, at least 92%, at least 93%, at least 94%, at least 95%, at least 96%, at least 97%, at least 98%, or at least 99% nucleic acid sequence identity with the nucleic acid sequence of SEQ ID NO: 22.

### 2.3 Linkers and Self-Cleaving Peptides in Bicistronic and Tricistronic Transgenes

In some embodiments in which a transgene encodes multiple interferon proteins, the proteins are translated as a polyprotein and individual IFN polypeptides are separated by a self-cleaving peptide. For example, the nucleic acid sequence encoding a first interferon polypeptide and the sequence encoding a second interferon polypeptide may be connected by a sequence encoding a first self-cleaving peptide. Optionally a sequence encoding a linker (e.g., Gly-Ser-Gly) is upstream (5′) from the sequence encoding the self-cleaving peptide. In some embodiments, the sequence encoding a second interferon polypeptide and the sequence encoding a third interferon polypeptide are connected by a sequence encoding a second linker peptide and a sequence encoding a second self-cleaving peptide. In some embodiments, the sequence encoding a second self-cleaving peptide is 3-prime from the sequence encoding a second linker peptide.

Suitable self-cleaving peptides include a 2A self-cleaving peptide, such as a P2A self-cleaving peptide, a T2A self-cleaving peptide, a F2A self-cleaving peptide, or an E2A self-cleaving peptide. See, e.g., Liu et al., “Systematic comparison of 2A peptides for cloning multi-genes in a polycistronic vector”. Sci Reports 7(1): 2193 (2017). In some embodiments, the first self-cleaving peptide is a P2A self-cleaving peptide and has the sequence of SEQ ID NO: 17.

In some embodiments, a first self-cleaving peptide and a second self-cleaving peptide are the same. For example, in some embodiments, both are P2A. In some embodiments, a first self-cleaving peptide and a second self-cleaving peptide are not the same. For example, in some embodiments, a first self-cleaving peptide is a P2A self-cleaving peptide and a second self-cleaving peptide is a T2A self-cleaving peptide. In some embodiments, the second self-cleaving peptide is a T2A self-cleaving peptide and has the sequence of SEQ ID NO: 18.

### 2.4 Transcriptional Regulatory Elements

In some embodiments, the rAAV vector described herein comprises transcriptional regulatory elements such as post-transcriptional regulatory elements, transcription initiation and termination sequences, efficient RNA processing signals such as polyadenylation (polyA) signals, leader sequences, and ribosomal binding sites. The rAAV vector may contain none, one or more of any of the elements described herein.

In some embodiments, the rAAV vector and expression cassette described herein comprise post-transcriptional regulatory elements.

### Woodchuck Hepatitis Virus Posttranscriptional Regulatory Element

In one embodiment, the post-transcriptional regulatory element is woodchuck hepatitis virus posttranscriptional regulatory element (WPRE). The WPRE is characterized and described in U.S. Pat. Nos. 6,136,597, and 6,287,814 incorporated herein by reference. As described therein, the WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. An examples of a sequence encoding a suitable WPRE is shown in SEQ ID NO: 23. In one embodiment, the transgene is located between the CAG promoter and the WPRE.

### Polyadenylation Signal

In some embodiments, the rAAV vector described herein comprises a polyadenylation (polyA) signal. In one embodiment, the rAAV vector comprises a polyA signal from SV40. In one embodiment, the rAAV vector comprises a polyA with the nucleic acid sequence of SEQ ID NO: 24. In other embodiments, the rAAV vector comprises a polyA signal from bovine growth hormone (bGH). Examples of other suitable polyA signals include, a synthetic polyA signal, a polyA from human growth hormone (hGH), rabbit beta-globin (RGB), or modified RGB (mRGB).

### Kozak Sequence

In some embodiments, a Kozak sequence (e.g., ATGATT; see, e.g., Kozak et al, Nuc Acids Res 15(20): 8125-8148 (1987)) is included between the CAG promoter and the transgene to enhance translation from the correct initiation codon.

### Variant Sequences Substantially Identical To A Reference Sequence Described Herein

It will be understood by those of skill in the art that regulatory sequences and protein sequences can tolerate a certain degree of variation whilst retaining the function or activity of the reference sequence. In certain approaches described herein in which a regulatory sequence, a coding sequence, or construct sequence (see annotated sequences of constructs, below) is called out, it is also contemplated that a substantially identical sequence that retains the function or activity of the called-out sequence may be used in its place. A substantially identical sequence is a sequence with at least about 90% sequence identity, preferably at least about 91%, at least about 95%, at least about 96%, at least about 97%, at least about 98%, or at least about 99% nucleic acid or polypeptide sequence identity, over the reference sequence. In one approach sequence identity shared by two sequences, or defined sequence segments, is determined using the local homology algorithm of Smith & Waterman, J. Mol. Bio 147 (1): 195-197 (1981); Adv. Appl. Math. 2:482 (1981). In one approach sequence identity shared by two sequences, or defined sequence segments, is determined using the method of Altschul et al., (1990) “Basic local alignment search tool.” J. Mol. Biol. 215:403-410 or computerized implementations of this method, such as the BLASTN or BLASTP programs available from the National Center for Biotechnology Information.

### 2.5 Adeno-Associated Viral Vector

Wildtype adeno-associated virus (AAV) is a member of the Parvovirus family. It is a small nonenveloped, icosahedral virus with a single-stranded linear DNA genome 4.7 kilobases (kb) in length. AAV is a member of the genus Dependovirus, because in its wildtype state, AAV depends on a helper virus (e.g. Adenovirus or Herpes simplex virus) to provide critical replication proteins, as AAV is naturally replication-defective. The 4.7-kb AAV genome is flanked on each end by two inverted terminal repeats (ITRs) that fold into hairpins important for genome replication. This characteristic of natural replication-defectiveness and the ability to transduce nearly every cell type in mammals, makes AAV an ideal therapeutic vector for use in gene therapy, genome editing and vaccine delivery. In the wildtype state, the AAV life cycle includes a latent phase wherein AAV genomes can site-specifically integrate into host chromosomes, and an infectious phase during which (following infection with a helper virus like adenovirus or herpes simplex virus) the integrated genomes are subsequently rescued, replicated, and packaged into infectious virions. When vectorized for use as an rAAV, the viral Rep and Cap genes of the AAV are removed and provided in trans during virus production, making the ITRs the only viral DNA that remains (A. Vasileva, et al., Nat Rev Microbiol 3: 837-847 (2005)). See also B. J. Carter, in “Handbook of Parvoviruses”, ed., P. Tijsser, CRC Press, pp. 155 168 (1990). Rep and Cap can then be substituted with a variety of different genome configurations to enable its use for gene therapy, genome editing or passive vaccines. These vectorized recombinant AAVs transduce both dividing and non-dividing cells, and show robust stable expression in quiescent tissues like the brain.

Numerous AAV serotypes are known (see, e.g., Wang et al., “Adeno-associated virus vector as a platform for gene therapy delivery.” Nat Rev Drug Discov 18: 358-378 (2019)) including naturally occurring serotypes such as AAV1, AAV2, AAV3, AAV4, AAVS, AAV6, AAV7, AAV8, AAV9 and others. In addition, numerous methods exist and are known to those in the art for engineering novel capsid serotypes (see, e.g., Wang et al., 2019, Id). See EP2573170 (“Adeno-associated virus (AAV) serotype 9 sequences, vectors containing same, and uses therefor”). Both naturally occurring and engineered capsid serotypes comprise characteristic tropisms for different species, organs, tissues, cell types, and functions. Each naturally occurring wildtype capsid serotype has a corresponding ITR sequence important for viral replication and packaging. In many cases, the genomic ITRs from one capsid serotype can be used to package a genome inside a different capsid serotype. ITRs can also be engineered to improve various characteristics important for therapeutic rAAV vectors. See Li, et al., “Engineering adeno-associated virus vectors for gene therapy.” Nat Rev Genet 21: 255-272 (2020).

Accordingly, AAV transfer vector genome constructs can be designed so that the AAV ITRs flank the transgene. rAAV vectors as delivery systems in gene therapy have been well described, e.g. in Dunbar, et al. “Gene therapy comes of age” Science 359:6372 (2018); Penaud-Budloo, et al., “Pharmacology of recombinant Adeno-Associated Virus production” Mol Ther Meth Clin Dev 8: 166-180 (2018); Gonçalves, M.A. “Adeno-associated virus: from defective virus to effective vector.” Virol J 2: 43 (2005); Li, et al “Engineering adeno-associated virus vectors for gene therapy.” Nat Rev Genet 21: 255-272 (2020); each of which is incorporated by reference for all purposes.

Exemplary rAAV vectors useful according to the disclosure include those with genomes existing in either single-stranded (ss) or self-complementary (sc) configurations. AAV sequences that may be used in the present invention can be derived from the genome of any AAV serotype or may further be engineered. In some embodiments, AAV1, AAV2, AAV3, AAV3b, AAV4, AAV5, AAV6, AAV7, AAV8, AAV-rh8, AAV9, AAV9-hu14, AAV10, AAV-rh10, AAV11, AAV12, AAV-NP22, AAV-NP66, AAV-NP40, AAV-NP59, AAV-DJ, AAV-DJ/8, AAV-LK03, AAV-rh74, or AAV-hu37, variants thereof, or AAVs yet to be discovered or variants thereof may be used for the rAAV vectors of the present invention. See, e.g., WO 2005/033321, which is incorporated herein by reference.

In some aspects, the rAAV vector is a single stranded (ss) rAAV vector. In some embodiments, the rAAV vector is a self-complementary (sc) vector. “Self-complementary rAAV” or “scAAV” refers to a vector having an expression cassette in which a coding region carried by a rAAV nucleic acid sequence has been designed to form an intra-molecular double-stranded DNA template. Upon transduction, rather than waiting for cell-mediated synthesis of the second strand, the two complementary halves of scAAV will associate to form one double-stranded DNA (dsDNA) unit that is ready for immediate replication and transcription. See, e.g., McCarty, et al., “Self-complementary AAV vectors: advances and applications”, Mol Ther 16: 1648-1656 (2008). Self-complementary AAVs are described in, e.g., U.S. Pat. Nos. 6,596,535; 7,125,717; and 7,456,683, each of which is incorporated herein by reference in its entirety.

AAV DNA ends comprise a 145-bp inverted terminal repeat (ITR) characterized by a T-shaped hairpin structure which becomes a 3′ hydroxyl group serving as a primer for the initiation of viral DNA replication (Berns K. “Parvovirus replication”, Microbiol Rev 54: 316-329 (1990)). The ITRs are the only sequences of viral origin needed to guide genome replication and packaging during vector production. See e.g. Gonçalves, M.A. “Adeno-associated virus: from defective virus to effective vector.” Virol J 2: 43 (2005).

Accordingly, in some embodiments, the rAAV vector comprises AAV inverted terminal repeats (ITRs) flanking the promoter and transgene sequences. The ITR sequences may be from any naturally occurring serotype or they may be engineered. In some embodiments, the ITR is AAV1 ITR, AAV2 ITR, AAV3 ITR, AAV4 ITR, AAV5 ITR, AAV6 ITR, AAV7 ITR, AAV8 ITR, AAV-rh8 ITR, AAV9 ITR, AAV10 ITR, AAV-rh10 ITR, AAV11 ITR, or AAV12 ITR, or variants thereof. In one embodiment, ITR sequences may be from AAV2 (GenBank Accession number AF043303). In some embodiments, full-length AAV ITRs are used. In some embodiments, a shortened version of the AAV ITRs, can be used in which the D-sequence and terminal resolution site (trs) are deleted (Ling et al., “Enhanced transgene expression from recombinant single-stranded D-sequence-substituted adeno-associated virus vectors in human cell lines in vitro and in murine hepatocytes in vivo.” J Virol 89(2): 952-961 (2015)). In some embodiments, ITRs are be selected to generate a single-stranded (ss) rAAV vector. In some embodiments, ITRs may be selected to generate a self-complementary rAAV vector, such as defined above.

In some aspects, the disclosure provides a recombinant adeno-associated virus (rAAV). The rAAV comprises an AAV capsid, and the rAAV vector as described herein. An AAV capsid is composed of 60 viral protein subunits, VP1, VP2, and VP3, that are arranged in an icosahedral symmetry in a ratio of approximately 1:1:10 for VP1:VP2:VP3. The AAV capsid can be of any AAV serotype. For example, the AAV capsid can be an AAV1, AAV2, AAV3, AAV3b, AAV4, AAV5, AAV6, AAV7, AAV8, AAV9, AAV10, AAV11, AAV12, or variants thereof. In one embodiment, the AAV capsid is an AAV9 capsid. In some embodiments, the AAV capsid can be from an engineered AAV. For example, the AAV capsid can be an AAV-rh8, AAV9-hu14, AAV-rh10, AAV-NP22, AAV-NP66, AAV-NP40, AAV-NP59, or variants thereof. The AAV ITRs may be of the same AAV origin as the capsid employed in the resulting recombinant AAV. For example, the rAAV vector may contain AAV2 genome ITRs and AAV2 capsid proteins. In other embodiments, the rAAV may be pseudotyped, where the ITRs are of one AAV serotype and the capsid proteins are of a different AAV serotype. For example, the rAAV vector may comprise two AAV2 ITRs and be encapsulated with the capsid proteins of AAV9. In some embodiments, the rAAV vector comprises two AAV2 ITRs and is encapsulated with the proteins of AAV1, AAV3, AAV3b, AAV4, AAV5, AAV6, AAV7, AAV8, AAV9, AAV10, AAV11, AAV12, AAV-rh8, AAV9-hu14, or AAV-rh10. In some embodiments, the AAV capsid is engineered to be chimeric, comprising sequences from two or more different AAV serotypes. For example, the AAV capsid can be an AAV-DJ, AAV-DJ/8, AAV-LK03, AAV-NP22, AAV-NP66, AAV-NP40, or AAV-NP59.

It will be understood by those of skill in the art that variants of AAV capsid proteins and AAV ITRs can be used to generate the rAAV of the present disclosure. The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like.

Suitable AAV proteins may be derived from “engineered AAV” with a non-naturally occurring capsid protein. Such an artificial capsid may be generated by any suitable technique, using a selected AAV sequence (e.g., a fragment of a VP1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid.

### 2.6 Producing Recombinant AAVs

The rAAV vectors described herein may be generated and isolated using methods known in the art. See, e.g., U.S. Pat. Nos. 7,790,449, 7,588,772, WO 2005/033321, and Zolotukin et al., “Production And Purification Of Serotype 1, 2, And 5 Recombinant Adeno-Associated Viral Vectors.” Methods 28:158-167 (2002), incorporated by reference, and Penaud-Budloo et al., 2018; Gonçalves, M.A. “Adeno-associated virus: from defective virus to effective vector.” Virol J 2: 43 (2005); Li, et al “Engineering adeno-associated virus vectors for gene therapy.” Nat Rev Genet 21: 255-272 (2020); all incorporated by reference and cited above. For general methods on genetic and recombinant engineering, recombinant engineering, and transfection techniques see e.g., Sambrook et al, Molecular Cloning: A Laboratory Manual, Cold Spring Harbor Press, Cold Spring Harbor, N.Y.; Graham et al., Virol., 52:456 (1973); Davis et al., Basic Methods in Molecular Biology, Elsevier, (1986); and Chu et al., Gene 13:197 (1981). In some embodiments, rAAVs may be produced using the triple transfection method (described in detail in U.S. Pat. No. 6,001,650). In one approach, a producer cell line is transiently transfected with the therapeutic rAAV plasmid construct described herein and both a plasmid that encodes Rep and Cap, as well as an adenoviral helper plasmid construct. In another approach, a packaging cell line that stably expresses Rep and Cap is then transiently transfected with the therapeutic rAAV plasmid described herein and an adenoviral helper plasmid. In yet another approach, a packaging cell line that stably expresses both Rep and Cap as well as adenoviral helper proteins is then transiently transfected with the therapeutic rAAV plasmid described herein. In some approaches, rAAVs are produced through live infection with either wildtype or engineered helper adenovirus or herpesvirus. In some approaches, necessary rAAV components are encoded from within one to three live baculoviruses and these are then used to infect insect cells such as those isolated from Spodoptera frugiperda (e.g. Sf9). In some approaches, infection with helper virus is not required and the helper functions can be supplied by transient transfection of the cells with constructs that encode the required helper functions, or the cells can be engineered to stably contain genes encoding the helper functions. Cells for producing rAAVs are known in the art and include, but are not limited to those capable of baculovirus infection, including insect cells such as High Five, Sf9, Se301, SelZD2109, SeUCR1, Sf9, Sf900+, Sf21, BTI-TN-5B1-4, MG-1, Tn368, HzAm1, BM-N, Ha2302, Hz2E5 and Ao38, and mammalian cells such as HEK293, HeLa, CHO, NSO, SP2/0, PER.C6, Vero, RD, BHK, HT 1080, A549, Cos-7, ARPE-19 and MRC-5 cells.

### 3. Cells

In one aspect, the invention provides a recombinant or isolated cell comprising an expression cassette or transgene described herein.

### 4. Pharmaceutical Compositions

Also provided herein are pharmaceutical compositions of the vectors of the invention. The pharmaceutical compositions described herein are designed for delivery to subjects in need thereof by any suitable route or a combination of different routes. In some embodiments, the pharmaceutical composition comprising the rAAV vector, the rAAV, or the isolated cell as described herein further comprises a pharmaceutically acceptable excipient or carrier. In some approaches, sterile injectable solutions can be prepared with the rAAV vectors in the required amount and an excipient suitable for injection into a human patient. In some embodiments, the pharmaceutically and/or physiologically acceptable excipient is particularly suitable for administration to the brain. For example, a suitable carrier may be buffered saline or other buffers, e.g., HEPES, to maintain pH at appropriate physiological levels, stabilizing agents, adjuvants, diluents, or surfactants. For injection, the excipient will typically be a liquid. Exemplary pharmaceutically acceptable excipients include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution.

### 5. Administration Methodology and Dose

### 5.1 Routes of Administration and Injection Methods

Aspects of the invention include methods of administering the rAAV of the present disclosure for treating cancer in a subject in need of treatment. In some approaches, the administration includes administering an rAAV vector, the isolated cell, or the pharmaceutical composition to a subject. Administration is not limited to a particular site or method. Any suitable route of administration or combination of different routes can be used, including systemic administration (e.g., intravenous, intravascular, intraarterial), local injection into the central nervous system (CNS; e.g. intratumoral injection, intracranial injection, intracerebral injection, intracerebroventricular, or injection into the Cerebrospinal fluid (CSF) via the cerebral ventricular system, cisterna magna, or intrathecal space), or local injection at other bodily sites (e.g. intraocular, intramuscular, subcutaneous, intradermal injection, transdermal).

In some aspects, intracerebroventricular injection occurs in the right lateral ventricle, left lateral ventricle, third ventricle, fourth ventricle, interventricular foramina (also called the foramina of Monro), cerebral aqueduct, central canal, median aperture, right lateral aperture, left lateral aperture, perivascular space, or the subarachnoid space.

Administration can be performed by use of an osmotic pump, by electroporation, or by other means. In some approaches, administration of the rAAV of the present disclosure can be performed before, after, or simultaneously with surgical tumor removal or biopsy.

### Convection Enhanced Delivery

In one approach, the rAAV vector is delivered by Convection Enhanced Delivery (CED). CED uses direct infusion of a drug-containing liquid into tissue so that transport is dominated by convection. The method of CED has been described in detail for example in Ung et al., “Convection Enhanced Delivery for glioblastoma: Targeted delivery of antitumor therapeutics”, CNS Oncol 4(4): 225-234 (2015), and Jahangiri et al., “Convection Enhanced Delivery in glioblastoma: A review of preclinical and clinical studies”, J Neurosurg 126(1): 191-200 (2017). Any convection-enhanced delivery device may be appropriate for use. In some embodiments, the device is an osmotic pump. In some embodiments, the device is an infusion pump. In some approaches, CED is performed with a step-design cannula.

In some embodiments, magnetic resonance imaging (MRI) guided CED is performed to deliver the rAAV vectors of the present disclosure. In some embodiments, CED further comprises the use of a tracing agent. In one aspect, the tracing agent is an MRI contrast enhancing agent. In one approach, the MRI contrast enhancing agent is gadolinium and related chemical derivatives. In some embodiments, the MRI contrast enhancing agent and the rAAV are administered simultaneously. In some embodiments, the MRI contrast enhancing agent is mixed with the rAAV directly prior to administration.

### 5.2 Dosage and Effective Amounts

Dosage values may depend on the nature of the product and the severity of the condition. It is to be understood that for any particular subject, specific dosage regimens can be adjusted over time and in course of the treatment according to the individual need and the professional judgment of the person administering or supervising the administration of the compositions. Accordingly, dosage ranges set forth herein are exemplary only and are not intended to limit the scope or practice of the claimed composition.

The amount of rAAV administered will be an “effective amount” or a “therapeutically effective amount,” i.e., an amount that is effective, at dosages and for periods of time necessary, to achieve a desired result. A desired result would include an improvement in interferon expression or activity in a target cell, reduction in tumor size and/or tumor growth, prolonged survival or a detectable improvement in a symptom associated with cancer that improves patient quality of life. Alternatively, if the pharmaceutical composition is used prophylactically, a desired result would include a demonstrable prevention of one or more symptoms of cancer. A therapeutically effective amount of such a composition may vary according to factors such as the disease state, molecular tumor profile (e.g. tumor mutation types), age, sex, and weight of the individual, or the ability of the viral vector to elicit a desired response in the individual. Dosage regimens may be adjusted to provide the optimum response. A therapeutically effective amount is also one in which any toxic or detrimental effects of the viral vector are outweighed by the therapeutically beneficial effects.

Quantification of genome copies (GC), vector genomes (VG), virus particles (VP), or infectious viral titer may be used as a measure of the dose contained in a formulation or suspension. Any method known in the art can be used to determine the GC, VG, VP or infectious viral titer of the virus compositions of the invention, including as measured by qPCR, digital droplet PCR (ddPCR), UV spectrophotometry, ELISA, next-generation sequencing, or fluorimetry as described in, e.g. in Dobkin et al., “Accurate Quantification and Characterization of Adeno-Associated Viral Vectors.” Front Microbiol 10: 1570-1583 (2019); Lock et al., “Absolute determination of single-stranded and self-complementary adeno-associated viral vector genome titers by droplet digital PCR.” Hum Gene Ther Methods 25: 115-125 (2014); Sommer, et al., “Quantification of adeno-associated virus particles and empty capsids by optical density measurement.” Mol Ther 7: 122-128 (2003); Grimm, et al. “Titration of AAV-2 particles via a novel capsid ELISA: packaging of genomes can limit production of recombinant AAV-2.” Gene Ther 6: 1322-1330 (1999); Maynard et al., “Fast-Seq: A Simple Method for Rapid and Inexpensive Validation of Packaged Single-Stranded Adeno-Associated Viral Genomes in Academic Settings.” Hum Gene Ther 30(6): 195-205 (2019); Piedra, et al., “Development of a rapid, robust, and universal picogreen-based method to titer adeno-associated vectors.” Hum Gene Ther Methods 26: 35-42 (2015); which are incorporated herein by reference. An exemplary human dosage range in vector genomes per kilogram bodyweight (vg/kg) may be 10e6 vg/kg-10e15/kg vg per injection in a volume of 1-100,000 μl. An exemplary mouse dosage range may be 10e6 vg/kg-10e15/kg vg per injection in a volume of 1-1000 μl. An exemplary dog dosage range may be 10e6 vg/kg-10e15/kg vg per injection in a volume of 1-10,000 μl.

In one approach, the composition is administered in a single dosage selected from those above listed. In another embodiment, the method involves administering the compositions in two or more dosages (e.g., split dosages). In another embodiment, multiple injections are made at different locations. In another embodiment, a second administration of an rAAV is performed at a later time point. Such time point may be weeks, months or years following the first administration. In some embodiments, multiple treatments may be required in any given subject over a lifetime. Such additional administration is, in one embodiment, performed with an rAAV having a different capsid serotype than the rAAV from the first or previous administration. In another embodiment, such additional administration is performed with an rAAV having the same capsid serotype as the rAAV from the first or previous administration.

### 5.3 Combination Therapies

In some approaches, the rAAV vectors of the present disclosure are used in combination with one or more additional anti-cancer agents and/or therapies, including any known, or as yet unknown, anti-cancer agent or therapy which helps preventing development of, slowing progression of, reversing, or ameliorating the symptoms of cancer, e.g., .glioblastoma. The one or more additional anti-cancer agents and/or therapies may be administered and/or performed before, concurrent with, or after administration of the rAAV vectors described herein. The combined administration includes co-administration, using separate formulations or a single pharmaceutical formulation. In some embodiments, the rAAV vectors of the present disclosure are used in combination with one or more anticancer therapies, such as chemotherapy, radiation therapy, tumor treating field (TTF) therapy, immunotherapy, and surgical treatment.

In one embodiment, the rAAV vectors of the present disclosure are used in combination with a chemotherapy that involves temozolomide (TMZ). In one embodiment, the rAAV vectors of the present disclosure may be administered in combination with radiation therapy. In yet another embodiment the rAAV vectors may be administered in combination with radiation therapy and TMZ. Other chemotherapeutic agents that may be used in combination with the rAAV vectors include cyclophosphamide, docetaxel, hydroxydaunorubicin, adriamycin, doxorubicin, vincristine, and prednisolone.

In some embodiments, the rAAV vectors of the present disclosure are used in combination with an antiangiogenic therapy. Such antiangiogenic therapy may, for example, include the use of bevacizumab. In some embodiments, the rAAV vectors may be administered in combination with bevacizumab and TMZ.

In some approaches, the rAAV vectors of the present disclosure are used in combination with immunotherapy, for example a checkpoint inhibitor, such as ipilimumab, nivolumab, pembrolizumab, atezolizumab, avelumab, or durvalumab.

Examples of other anti-cancer agents that can be combined with the rAAV vectors include, without limitation any one or more of a kinase inhibitor, a co-stimulation molecule blocker, an adhesion molecule blocker, an anti-cytokine antibody or functional fragment thereof, a corticosteroid, a non-steroidal anti-inflammatory agent, a nitrogen mustard, an aziridine, an alkyl sulfonate, a nitrosourea (e.g. carmustine, semustine, lomustine, nimustine, or fotemustine), a non-classical alkylating agent, a folate analog, a purine analog, an adenosine analog, a pyrimidine analog, a substituted urea, an antitumor antibiotic, an epipodophyllotoxin, a microtubule agent, a camptothecin analog, a cytokine, a monoclonal antibody, a recombinant toxin, an immunotoxin, a cancer gene therapy, a cancer cell therapy, an oncolytic viral therapy, or a cancer vaccine.

In some embodiments, the rAAV vectors of the present disclosure are used in combination with a medical device such as Optune.

6.

### 6.1 Human Patients

Methods and gene therapy constructs disclosed herein may be used to treat patients with cancers and other diseases responsive to interferon treatment. In particular the invention may be used to treat patients with a glioma, such as a grade Ill or grade IV gioma (glioblastoma).

### 6.2 Canine Applications

The gene therapy methods and constructs disclosed herein may be used in treatment of animals as well as human patients. For example, as demonstrated in Example 5, below, a vector encoding a canine IFNβ protein was effective in an orthotopic canine patient-derived xenograft (PDX) mouse model of glioblastoma.

### 7. EXAMPLES

### 7.1 Example 1. Approach and Methods

rAAV Production

Recombinant AAV vector products were manufactured at SignaGen using a Ca₃(PO₄)₂ transient triple transfection protocol in adherent human HEK293 AAV-HT™ cells, followed by double cesium chloride density gradient purification, desalting, filter sterilization, and qPCR titering. Plasmids included: pAAV helper (SignaGen), transfer vectors we developed (ssAAV-CAG-IFNβ with AAV2 ITRs), and SignaGen's pseudotyping plasmid for AAV9 (AAV2 Rep, AAV9 Cap) (SignaGen). All vectors were confirmed free of endotoxins using a limulus amebocyte lysate assay.

### In Vitro Vector Expression Confirmation by IFNβ ELISA

To validate functional expression of IFNβ prior to use in animals, an IFNβ ELISA was used on media collected following cell transduction experiments. Assays used a human IFNβ ELISA kit (Thermo Fisher Cat #414101), a mouse IFNβ ELISA kit (Thermo Fisher Cat #424001) following the manufacturer's protocol. Cells were maintained in RPMI 1640 media (Gibco Cat #11875) supplemented with 10% FBS and 1% antibiotic/antimycotic. In all cell types, 1-hr prior to transduction at 80% confluency, media was changed, then 40K cells/condition were transduced with the respective ssAAV9-CAG-IFNβ vector (human/mouse) diluted in dPBS at MOI 1M. The following day the media was replaced to remove vectors that did not transduce and the media was not changed again. IFNβ levels were measured 2-days post-AAV administration using a Molecular Devices plate reader at 450 nm. Experiments were performed in technical triplicate and corrected for background by subtracting the signal from the PBS-diluent negative control wells.

### Fast-Seq Validation of Packaged Viral Genomes

Total packaged gDNA was extracted from 1E11 full rAAV particles for each rAAV9 vector lot. AAV genome libraries were prepared following a Tn5 tagmentation-based protocol called Fast-Seq described separately (Maynard et al., “Fast-Seq, a universal method for rapid and inexpensive genomic validation of rAAV vectors in preclinical settings v1 (protocols.io.utzewp6)”; Maynard et al. “Fast-Seq, a simple method for rapid and inexpensive validation of packaged ssAAV genomes in academic settings”, Hum. Gene Ther. Methods).

Each adapter contained a 12-nucleotide unique barcode for identifying samples after multiplexing. The resulting library was diluted to 10-pM in 600-mL of HT1 hybridization buffer (Illumina Nextera XT kit Cat #FC-131-1024) and 10-mL was loaded onto a 300-cycle MiSeq Nano v2 flow cell (Illumina Cat #MS-102-2002) for paired-end 2×75-bp sequencing. Resultant reads were demultiplexed using Illumina's bcl2fastq v2.19.0.316. Data were returned in fastq format and filtered using Trimmomatic (Bolger et al. (2014), “Trimmomatic: a flexible trimmer for Illumina sequence data”, Bioinformatics 30, 2114-2120) to remove adapter sequences, low quality reads (PHRED score <30, or length <50-bp), unmapped and unpaired reads. Trimmed reads were then aligned to the rAAV transfer vector plasmid reference sequence with BWA v0.7.17 (Li & Durbin (2009), “Fast and accurate short read alignment with Burrows-Wheeler transform”, Bioinformatics 25, 1754-1760), using the mem algorithm. Alignments were saved as BAM files, which were then used to generate VCF files using GATK Haplotype Caller algorithm (McKenna et al. (2010), “The Genome Analysis Toolkit: a MapReduce framework for analyzing next-generation DNA sequencing data”, Genome Res. 20, 1297-1303). SNPs and indels identified in VCF files were filtered using BCFtools filter algorithm, with a 15× depth threshold and a 90% allele fraction requirement. A consensus sequence was generated using BCFtools consensus algorithm (Danecek & McCarthy (2017), “BCFtools/csq: haplotype-aware variant consequences”, Bioinformatics 33, 2037-2039). Alignment and fragment distribution statistics were obtained with Picard tools (https://broadinstitute.github.io/picard/). Coverage spanned 100% of the rAAV transfer vector genome reference sequence, including the ITRs. Sequencing validated that genomic payload sequence from all vectors were full-length and lacked genomic rearrangements.

### Animals

Adult 5-6-week-old female athymic nude mice (homozygous nu/nu) were purchased from Harlan Laboratories (Cat #490, Livermore, CA) as recipients for xenografts. Adult 5-6-week-old female C57BL/6 mice (C57BL/6NCrl) were purchased from Harlan Laboratories (Cat #027, Livermore, CA) as recipients for allografts. All mice were housed under specific-pathogen-free housing conditions and were given continual access to food and water ad libitum. The Institutional Animal Care and Use Committee of UCSF approved all mouse procedures.

### Human Tissue

We obtained primary fresh human adult female glioblastoma patient tumor tissue from the UCSF Tissue Bank IRB #10-01318 through Principal Investigator Joanna Phillips.

### Primary Human Glioblastoma Tumor Sample Acquisition

An adult female patient was taken to the operating room for a fronto-parietal parasagittal craniotomy for tumor resection. The preoperative stereotactic magnetic resonance images (MRI) were registered to physical space using the stereotactic neuronavigation system (BrainLab®, Munich, Germany). The accuracy of the registration was confirmed using anatomic landmarks. Surgical resection of the tumor was carried out in the usual manner. Throughout the resection, fresh tissue samples were obtained. The location of each sample (including a screenshot and DICOM coordinates) was collected using the stereotactic probe immediately prior to collecting the sample. Once collected from the single tumor, each of the six regional samples were immediately passed off the surgical field and placed into liquid nitrogen for subsequent analysis.

### Primary Human Glioblastoma Cell Culture

Primary human glioblastoma cells (SF11411 cells) obtained as described above were maintained at 37° C. in a 5% CO2 atmosphere with 21% oxygen, and grown in a 1:1 ratio of DMEM/F12 (Life Technologies, Carlsbad, CA) and Neurobasal medium (Life Technologies) supplemented with 5% FBS (Life Technologies), B-27 supplement without vitamin A (Life Technologies), N-2 supplement (Life Technologies), 1× GlutaMAX (Life Technologies), 1 mM NEAA (Life Technologies), 100 U/mL Anti-Anti (Life Technologies), 20 ng/mL EGF (R&D systems, Minneapolis, MN), 20 ng/mL FGF2 (Peprotech, Rocky Hill). Cell lines were validated using short-tandem repeat profiling at the UCSF Clinical Cancer Genomics Laboratory.

### Intracranial Orthotopic Tumor Establishment in Mice

All procedures were carried out under sterile surgical conditions. Mice were anesthetized by intraperitoneal injection of a mixture containing ketamine (100-mg/kg) and xylazine (10-mg/kg). The scalp was swabbed with 2% chlorhexidine, 20-30-μl of 0.25% bupivacaine was injected into the intra-cutaneous space of the scalp, and a skin incision ˜15-mm in length was made over the middle frontal to parietal bone. The surface of the skull was exposed so that a small hole could be made with a 25-gauge needle 3-mm to the right from bregma on top of the coronal suture. A 26-gauge needle attached to a Hamilton syringe was inserted into the hole in the skull. The needle was covered with a sleeve that limits the depth of the injection to 3.5-mm. In all cases (see below for specifics to each cell type), 300K cells in a 3-μl suspension were injected very slowly (˜3-μl/minute) by hand and then the needle was removed. The skull surface was swabbed with hydrogen peroxide before the hole was sealed with bone wax to prevent reflux. The scalp was closed with surgical staples. In all cases, treatment or vehicle negative controls included 10-15 similarly transplanted mice treated with either AAV9-GFP or vehicle (dPBS). All mice with FLuc^{pos} tumors were imaged 1-2 times/week and monitored for survival. Mice with FLuc^{neg} tumors were only monitored for survival.

For syngeneic orthotopic mouse allografts, immortalized male donor mouse GL261-FL glioma tumor cells (Szatmári et al. (2006), “Detailed characterization of the mouse glioma 261 tumor model for experimental glioblastoma therapy”, Cancer Sci. 97, 546-553) were acquired from the NCI Tumor Repository (Frederick, MD). Cells were grown in RPMI-1640 with 10% FBS and 1% penicillin-streptomycin in a humidified atmosphere of 5% CO₂. 300K tumor cells were injected intracranially into 10-15 anesthetized C57BL/6 recipients in a volume of 3-μL. 5-days post-transplant, once the tumor growth was in log-phase, AAV9-mIFNβ was administered via CED.

For orthotopic human xenografts, immortalized donor human GBM6-FL tumor cells from a 65-year-old male (Sarkaria et al. (2006), “Use of an orthotopic xenograft model for assessing the effect of epidermal growth factor receptor amplification on glioblastoma radiation response”, Clin. Cancer Res. 12, 2264-2271; Griffero et al. (2009), “Different response of human glioma tumor-initiating cells to epidermal growth factor receptor kinase inhibitors”, J. Biol. Chem. 284, 7138-7148) were a gift from Dr. David James at UCSF. Cells were grown and passaged in RPMI-1640 containing 10% FBS and 1% penicillin-streptomycin in a humidified atmosphere of 5% CO₂. 300K tumor cells were injected intracranially into 10-15 anesthetized athymic recipients in a volume of 3-μL. 9-days post-transplant, once the tumor growth was in log-phase, AAV9-hIFNβ was administered via CED.

For orthotopic human patient-derived xenografts, primary human female glioblastoma tumor cells were used. Cells were grown in RPMI-1640 with 10% FBS and 1% penicillin-streptomycin in a humidified atmosphere of 5% CO₂. 300K tumor cells were injected intracranially into 10 anesthetized athymic recipients in a volume of 3-μL. 7-days post-transplant, once the tumor growth was in log-phase, AAV9-hIFNβ was administered via CED.

For orthotopic canine patient-derived xenografts, primary canine J3Tbg tumor cells were obtained from a male beagle with a grade III astrocytoma (Dickinson et al. (2016), “Chromosomal Aberrations in Canine Gliomas Define Candidate Genes and Common Pathways in Dogs and Humans”, J. Neuropathol. Exp. Neurol. 75, 700-710). Cells were grown in DMEM with 10% FBS and 1% penicillin-streptomycin in a humidified atmosphere of 5% CO₂. 300K tumor cells were injected intracranially into 15 anesthetized athymic recipients in a volume of 3-μL. 2-days post-transplant, once the tumor growth was in log-phase, AAV9-cIFNβ was administered via CED.

### Convection-Enhanced Delivery (CED) AAV Administration Procedure

Just before treatment initiation, animals were randomized to treatment groups of 10 to 16 mice each. Mice were anesthetized with an intraperitoneal injection of ketamine/xylazine as described above. The scalp was cleaned with 2% chlorhexidine and a skin re-incision ˜10-mm in length was made over the middle frontal to parietal bone. The surface of the skull was exposed so that the hole made for tumor implantation was exposed. The CED brain infusion cannula was lowered through this hole into the tumor. The syringe was loaded with sample (saline, AAV9-GFP or AAV9-IFNβ), and attached to a microinfusion pump (Bioanalytical Systems, Lafayette, Ind.). An external microinfusion pump was used to drive fluid slowly (1-μL/min) into the glioblastoma tumor through the brain infusion cannula made of silica tubing (Polymicro Technologies, Phoenix, AZ) fused to a 0.1-ml syringe (Plastic One, Roanoke, VA) with a 0.5-mm stepped tip needle that protruded from the silica guide base. Samples (saline or AAV) were infused at a rate of 1-μL/min until the desired dose (1.89E11 vg or 1.89E12 vg in a volume of 10-15-μL) had been delivered. The brain infusion cannula was removed 2-min after infusion completion. The skull was swabbed with hydrogen peroxide and the hole was covered with bone wax before closing the scalp with staples.

### Live In Vivo Transduction Analysis by Firefly Luciferase Imaging and Quantitation in Mice

All mice with FLuc+ tumors were imaged non-invasively 1-2×/week on a Xenogen IVIS Spectrum imaging system (Caliper Life Sciences). D-luciferin substrate (Biosynth Cat #L-8220) was administered at 120-mg/kg in saline by intraperitoneal injection with a 1-cc insulin syringe. Images were acquired 10-min after luciferin administration under inhalation isoflurane anesthesia. Living Image v4.5 software was used for image analysis and average radiance was quantified in p/s/cm²/sr. All mice in each experiment are shown on the same non-individualized radiance scale to enable accurate comparisons of bioluminescent intensity.

iPSC and Glioblastoma Organoid Culture

Human induced pluripotent stem cells (iPSCs) from the H28126 line (Pollen et al. (2019), Establishing Cerebral Organoids as Models of Human-Specific Brain Evolution”, Cell 176, 743-756.e17) were maintained using feeder-free conditions on Matrigel (BD Cat #354234) coated dishes in TeSR (Stem Cell Technologies Cat #85850) medium. iPSCs were differentiated using a modified Sasai organoid protocol (Kadoshima et al. (2013), “Self-organization of axial polarity, inside-out layer pattern, and species-specific progenitor dynamics in human ES cell-derived neocortex”, Proc. Natl. Acad. Sci. U.S.A 110, 20284-20289) for directed telencephalon differentiation. iPSCs were dissociated using Accutase (Stem Cell Technologies Cat #07920) and aggregated into 96 well v-bottom low adhesion plates (S-bio Cat #MS-9096VZ). Aggregates were cultured in media containing Glasgow-MEM, 20% Knockout Serum Replacer, 0.1 mM NEAA, 1 mM sodium pyruvate, 0.1 mM b-ME, 100 U/ml penicillin/streptomycin and supplemented with Rho Kinase, Wnt and TGFβ inhibitors, 20-μM Y-27632 (Tocris), 3-μM IWR-1-endo (Cayman Cat #13659), and 5-μM SB431542 (Tocris Cat #1614). Rho Kinase inhibitor was removed after 6 days. Media was changed every other day throughout differentiation. After 18 days, organoids were transferred into 6 well low-adhesion plates in media containing DMEM/F12 with 1× Glutamax, 1× N2, 1× Lipid Concentrate, and 100 U/mL penicillin/streptomycin. After five weeks, organoids are matured in media containing DMEM/F12 with Glutamax, 1× N2, 1× Lipid Concentrate, 100 U/ml penicillin/streptomycin, 10λ Fetal Bovine Serum (Hyclone), 5-μg/ml heparin and 0.5% Growth factor-reduced Matrigel. After 10 weeks the concentration of Matrigel is increased to 1% and the media is additionally supplemented with 1× B-27.

### Statistics

Statistical analyses for in vivo survival curves were conducted with Prism v8 software. Experimental differences were evaluated using the Log-rank (Mantel-Cox) test. P values <0.05 were considered statistically significant. * P≤ 0.05, ** P≤ 0.01, *** P≤ 0.001, *** P≤ 0.0001.

### 7.2 Example 2. AAV9-mIFNβ Inhibits Tumor Growth and Increases Overall Survival in an Orthotopic Syngeneic Mouse Allograft Model of Glioblastoma

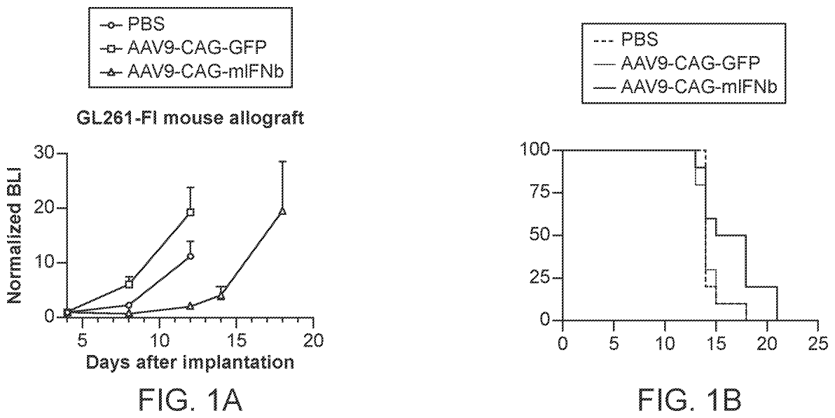

Wildtype C57BI6/J mice were treated with AAV9-mIFNβ, AAV9-GFP, or PBS via CED 5 days after orthotopic GL261-Fluc tumor implantation and bioluminescence was measured. AAV9-mIFNβ treatment slowed tumor growth compared to the AAV9-GFP and PBS control groups (FIG. 1A).

AAV9-mIFNβ treatment significantly improved overall survival with a median overall survival of 16.5 days (after tumor implantation) compared to 14 days in the PBS control group (P≤ 0.05; FIG. 1B).

### 7.3 Example 3. AAV9-hIFNβ Inhibits Tumor Growth and Increases Overall Survival in an Orthotopic Human Xenograft Mouse Model of Glioblastoma

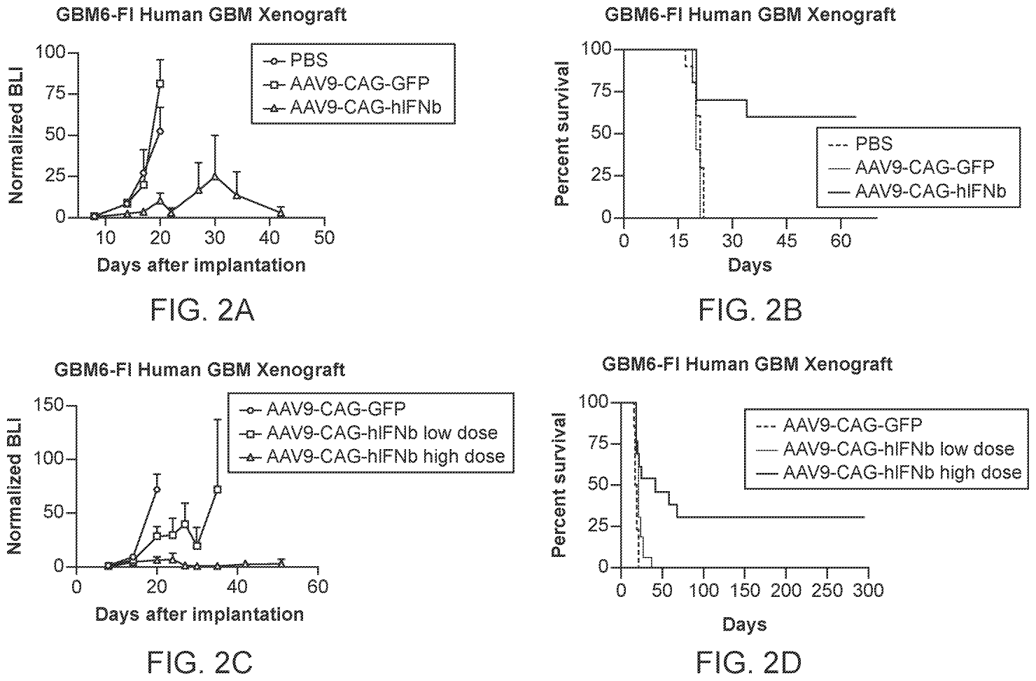

To assess the effectiveness of AAV9-hIFNβ treatment, athymic nude mice were treated with AAV9-hIFNβ, AAV9-GFP, or PBS via CED 9 days after orthotopic GBM6-Fluc tumor implantation and bioluminescence was measured 1-2 times per week. As shown in FIG. 2A, AAV9-hIFNβ treatment slowed tumor growth compared to the AAV9-GFP and PBS control groups.

The beneficial effect of AAV9-hIFNβ treatment was also reflected in overall survival with a median survival of 57 days (after tumor implantation) compared to 21 days in the PBS control group and 20 days in the AAV9-GFP control group (both P≤ 0.001; FIG. 2B).

To measure dose-dependent effects, athymic nude mice were treated with a high or low vector dose of AAV9-hIFNβ or AAV9-GFP via CED 9 days after orthotopic GBM6-Fluc tumor implantation and bioluminescence was measured 1-2 times per week. As shown in FIG. 2C, AAV9-hIFNβ treatment at both doses slowed tumor growth compared to the AAV9-GFP control group.

The beneficial effect of AAV9-hIFNβ treatment at both doses again resulted in significant improvements in overall survival with a median survival of 41 days in the high dose cohort (P≤ 0.0001), and 20 days in the low dose cohort (P<0.02) compared to 18 days in the AAV9-GFP control group (FIG. 2D).

### 7.4 Example 4. AAV9-hIFNβ Increases Overall Survival in an Orthotopic Primary Human Patient-Derived Xenograft (PDX) Mouse Model of Glioblastoma

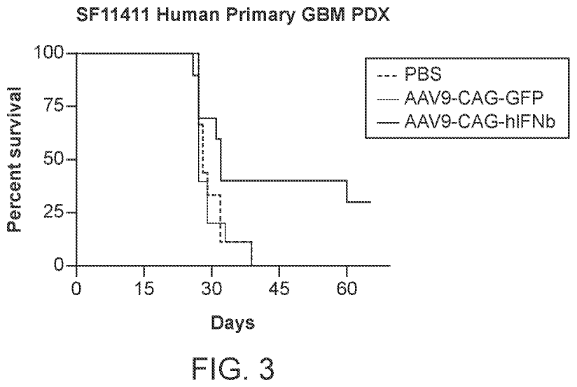

Athymic nude mice were treated with AAV9-hIFNβ, AAV9-GFP, or PBS via CED 7 days after orthotopic primary human patient tumor (SF11411) implantation. AAV9-hIFNβ treatment significantly improved overall survival with a median survival of 32 days compared to 27 days in the AAV9-GFP control group (P≤ 0.04; FIG. 3).

### 7.5 Example 5. AAV9-cIFNβ Increases Overall Survival in an Orthotopic Canine Patient-Derived Xenograft (PDX) Mouse Model of Glioblastoma

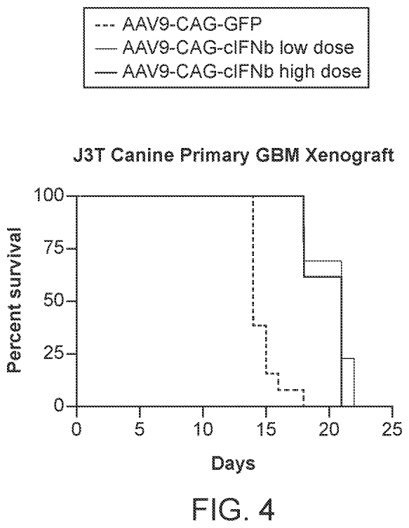

Athymic nude mice were treated with high or low vector doses of AAV9-cIFNβ, or AAV9-GFP via CED 2 days after orthotopic canine patient tumor (J3Tbg) implantation. Both high and low doses of AAV9-cIFNβ significantly improved overall survival with median survivals of 21 days each compared to 14 days in the AAV9-GFP control group (both P≤ 0.0001; FIG. 4).

### 7.6 Example 6. Reduction in Growth of GBM Tumor Spheroid Cultures

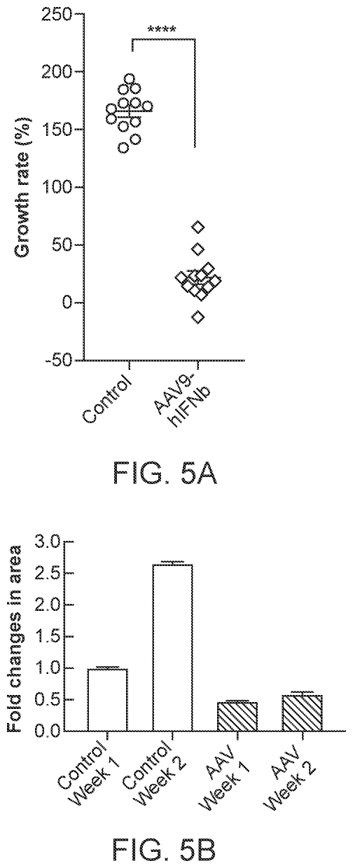

Growth rate was measured at 1 week and 2 week time points after treating with AAV9-hIFNβ or PBS. The GPMP004 cell line was transduced with mScarlet/luciferase. As determined by fluorescent microscopicopy, spheroids in the AAV9-hIFNβ treated condition began to shrink in week 1 after treatment, while spheroids in the control condition continued growing. Growth rate was significantly reduced in the AAV9-hIFNβ treated condition when compared to the control group (P≤ 0.0001; FIG. 5A).

Spheroid size (area) was measured at 1 week and 2 week time points after AAV9-hIFNβ or PBS treatment. As shown in FIG. 5B, from week 1 to week 2, fold changes in area were lower in the AAV9-hIFNβ treated condition than in the control condition.

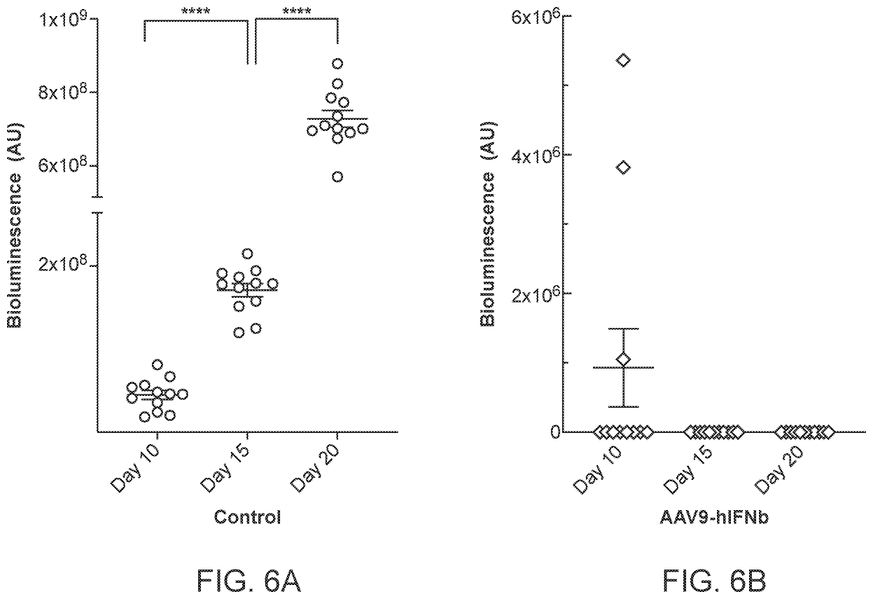

While measurements of bioluminescent signal from FIG. 6A in the control condition showed a significant increase from week 1 to week 2, and from week 2 to week 3 (both P≤ 0.0001; FIG. 6A), no increase was observed in the AAV9-hIFNβ treated condition, indicating inhibition of tumor growth by AAV9-hIFNβ (FIG. 6B).

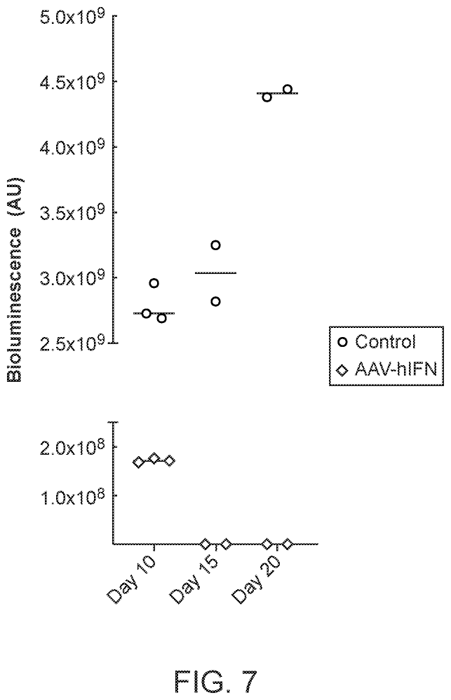

Proliferative activity of adherent human GBM tumor spheroid cultures at week 3 was observed. The GPMP004 cell line was transduced with mScarlet/luciferase. While tumor growth continued unabated in the PBS control condition, AAV9-hIFNβ treated tumors showed significantly decreased proliferative activity. FIG. 7 quantifies this response and demonstrates an increase in bioluminescent signal in the control condition, and a decrease in bioluminescent signal in the AAV9-hIFNβ treated condition at various time points.

### 7.7 Example 7. Coculture of Human Glioblastoma Cells Human Cerebral Organoids

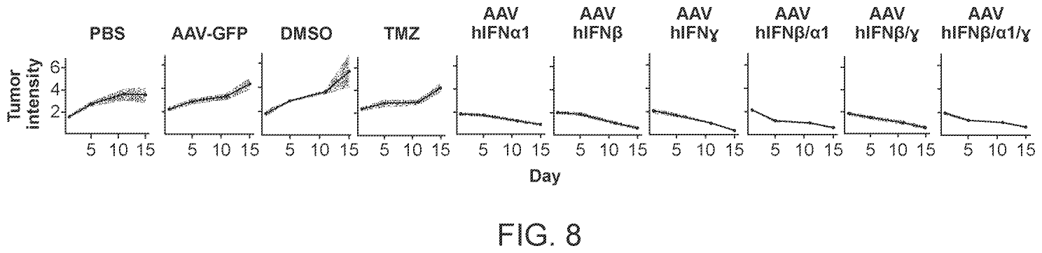

To study glioblastoma tumorigenesis and response to vectorized interferons, we co-cultured freshly resected human glioblastoma cells labeled with a red fluorescent protein (mScarlet), along with healthy human cerebral organoids composed of structurally complex pre-differentiated human pluripotent stem cell-derived astrocytes labeled with green fluorescent protein (GFP). Live confocal live imaging demonstrated that World Health Organization (WHO) grade IV human glioblastoma cells formed tumor spheres that invaded the healthy cerebral organoids, modeling glioblastoma behavior in vivo. To evaluate the relative cell intrinsic anti-tumor effects of our engineered IFN cytokines against relevant controls, a comparative time course of glioblastoma organoid co-cultures, monitored by live confocal imaging, was undertaken. See FIG. 8. All test and control vectors were packaged into AAV9. PBS, DMSO and AAV9-GFP had no significant effect on either glioblastoma tumor cells or healthy cerebral cells, thus the glioblastoma cells grew uncontrolled. TMZ chemotherapy (the current SOC) significantly decimated healthy cerebral cells and only slightly delayed glioblastoma tumor cell growth. In contrast, our platform of engineered cytokine payloads, composed of natural or engineered variants of IFNα1, IFNβ or IFN¥, either alone or in combination, were all able to reduce tumor size significantly and rapidly.

TABLE 5
Key to FIG. 8
(Showing sequence identifier)

| | |
|---|---|
| AAV-IFNβ | 25 |
| AAV-IFNα1 | 28 |
| AAV-IFNγ | 29 |
| AAV-IFNβ/α1 | 30 |
| AAV-IFNβ/γ | 31 |
| AAV-IFNβ/α1/γ | 32 |

### 7.8 Example 8. Human Glioblastoma Tumor Xenographs

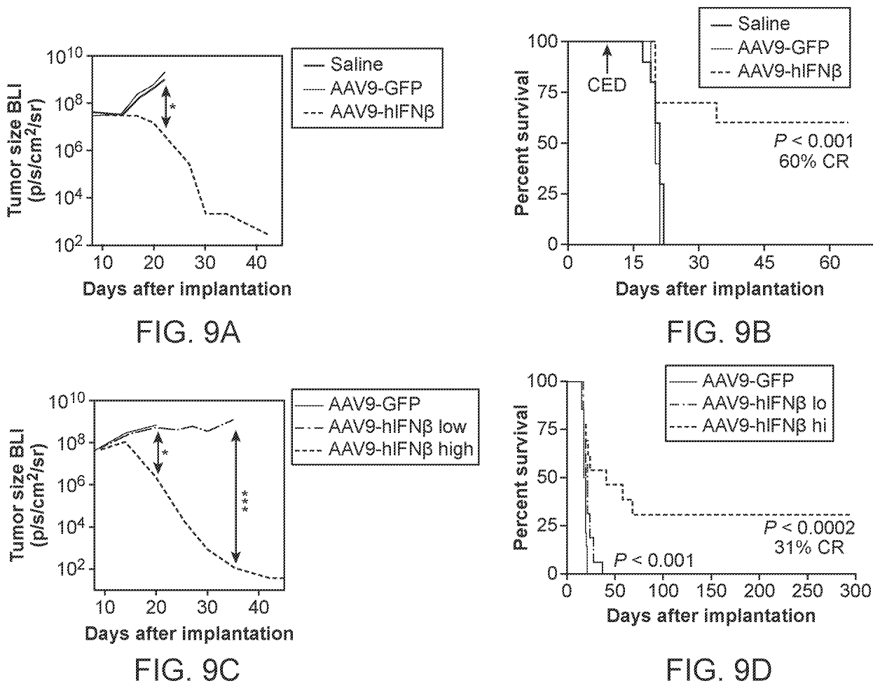

To assess long-term human glioblastoma tumor growth kinetics and overall survival following treatment in vivo, we again set up 3 treatment arms now comparing long-term responses in xenografts treated with saline, AAV9-GFP, or AAV9-hIFNβ. Thirty mice were injected intratumorally via CED (n=10 per treatment arm) and live imaged weekly for FLuc expression. Time course bioluminescent imaging (BLI) of the tumor revealed that human glioblastoma tumors grew rapidly when treated with the saline and AAV9-GFP controls, and shrank significantly (P<0.03) when treated with AAV9-hIFNβ (FIG. 9A). The live tumor BLI demonstrating rapid tumor shrinkage orthogonally validated the rapid tumor loss seen in our short-term immunohistopathology experiment seen previously. Correspondingly, Kaplan Meyer survival analysis demonstrated a significant improvement (P<0.001) in median overall survival (mOS) compared to control-treated animals (saline mOS 20 days; AAV9-GFP mOS 21 days) (FIG. 9B), with 60% of the treated xenografts experiencing a durable complete response (CR).

Having now established the safety and efficacy of our treatment paradigm, we next wanted to perform a long-term dose response study in these same GBM6-FL mice. To this end, we set up a new study with 3 treatment arms comparing responses in xenografts treated with AAV9-GFP, or AAV9-hIFNβ at two separate doses (1.89e11 vg and 1.89e12 vg). Forty-five mice were injected intratumorally via CED (n=15 per treatment arm) and live imaged weekly for FLuc expression. Time course tumor BLI again demonstrated that human glioblastoma tumors grew rapidly when treated with AAV9-GFP, and shrank significantly when treated with AAV9-hIFNβ (P<0.02-0.0004) (FIG. 9C), particularly at the high dose. Kaplan Meyer survival analysis demonstrated a significant improvement in mOS for both the low dose (mOS 20 days; P<0.001), and the high dose (mOS 41 days; P<0.0002), compared to control-treated animals (mOS 18 days) (FIG. 9D). Importantly, we saw durable CR in 31% of mice out to a year prior to harvest.

### 7.9 Example 9 Syngeneic Allografts

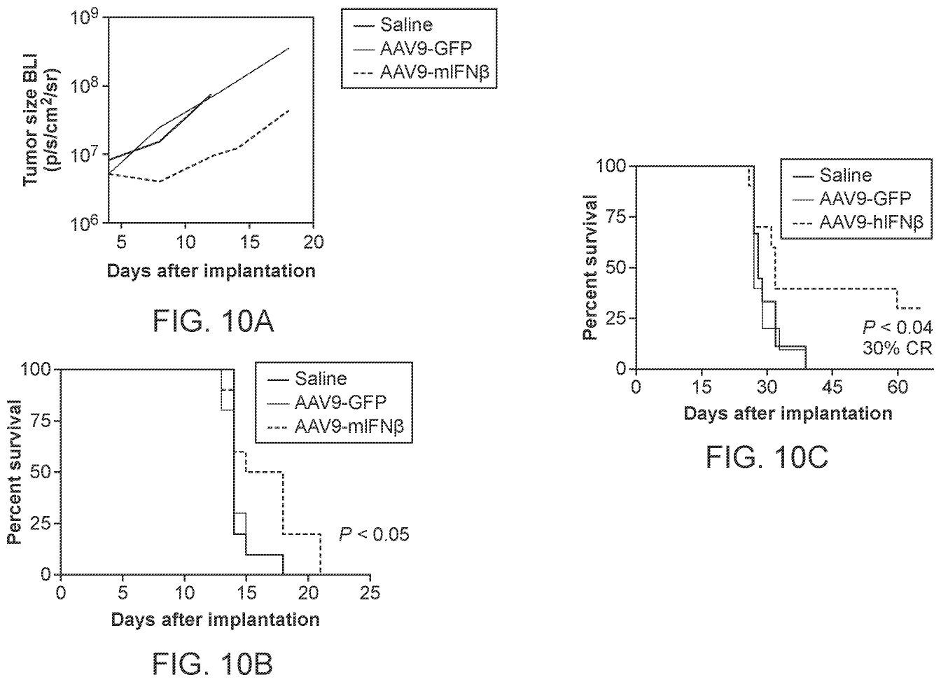

To assess murine glioblastoma tumor growth kinetics and overall survival following treatment in vivo, we set up a study with 3 treatment arms comparing responses in syngeneic allografts treated with saline, AAV9-GFP, or AAV9-mIFNβ. Thirty mice were injected intratumorally via CED (n=10 per treatment arm) and live imaged weekly for FLuc expression. Time course BLI tumor imaging revealed that mouse glioblastoma tumors grew rapidly when treated with the saline and AAV9-GFP controls, and were delayed in growth when treated with AAV9-mIFNβ (FIG. 10A). Kaplan Meyer survival analysis demonstrated an improvement in median overall survival (mOS 16.5 days) compared to control-treated animals (mOS 14 days in both saline and AAV9-GFP) but just missed reaching statistical significance (P<0.058) (FIG. 10B). Having now established the safety of our treatment paradigm in mouse models with complete immune systems, we next transitioned to patient-derived human xenograft models to enable assessment in unmodified primary human glioblastoma tumors in vivo.

### 7.10 Example 10. Human Patient-Derived Xenografts

Patient-derived xenografts (PDX) provided an opportunity to test our treatment paradigm in vivo in disease models composed of unmodified primary human glioblastoma tumor cells. Athymic nu/nu mice were transplanted with freshly resected primary human glioblastoma tumor cells and subsequently infused via intratumoral CED with our AAV9-hIFNβ vector. To assess overall survival following treatment in vivo, we set up 3 treatment arms comparing responses in PDX mice with tumors from an adult female with WHO grade IV glioblastoma, and treated with saline, AAV9-GFP, or AAV9-hIFNβ. Thirty mice were injected intratumorally via CED (n=10 per treatment arm) and monitored for symptoms and survival. Live tumor BLI as performed previously in engineered allografts and xenografts is not possible in this model since the primary tumors aren't engineered to express Fluc. Kaplan Meyer survival analysis demonstrated a significant improvement in mOS (32 days; P<0.04; 30% CR) compared to control-treated animals (saline mOS 28 days; AAV9-GFP mOS 27 days) (FIG. 10C).

### 7.11 Example 11. Primary Human Glioblastoma Cells Invade Glioblastoma Organoid

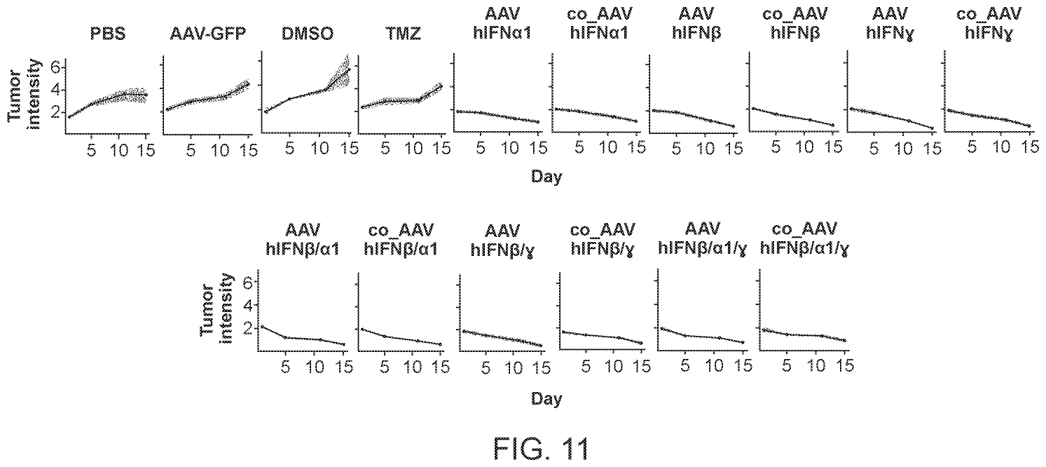

To study glioblastoma tumorigenesis and response to vectorized interferons, we co-cultured freshly resected human glioblastoma cells labeled with a red fluorescent protein (mScarlet), along with healthy human cerebral organoids composed of structurally complex pre-differentiated human pluripotent stem cell-derived astrocytes labeled with green fluorescent protein (GFP). Live confocal live imaging demonstrated that World Health Organization (WHO) grade IV human glioblastoma cells formed tumor spheres that invaded the healthy cerebral organoids, modeling glioblastoma behavior in vivo. To evaluate the relative cell intrinsic anti-tumor effects of our engineered IFN cytokines against relevant controls, a comparative time course of glioblastoma organoid co-cultures, monitored by live confocal imaging, was undertaken (FIG. 11). All test and control vectors were packaged into AAV9. PBS, DMSO and AAV9-GFP had no significant effect on either glioblastoma tumor cells or healthy cerebral cells, thus the glioblastoma cells grew uncontrolled. TMZ chemotherapy (the current SOC) significantly decimated healthy cerebral cells and only slightly delayed glioblastoma tumor cell growth. In contrast, our platform of engineered cytokine payloads, composed of natural or engineered variants of IFNα1, IFNβ or IFN¥, either alone or in combination, were all able to reduce tumor size significantly and rapidly.

TABLE 6
Key to FIG. 11
(Showing sequence identifier)

| | |
|---|---|
| AAV-IFNα1 | 28 |
| co_AAV-IFNα1 | 34 |
| AAV-IFNβ | 25 |
| co-AAV-IFNβ | 33 |
| AAV-IFNγ | 29 |
| co-AAV-IFNγ | 35 |
| AAV-IFNβ/α1 | 30 |
| co_AAV-IFNβ/α1 | 36 |
| AAV-IFNβ/γ | 31 |
| co_AAV-IFNB/γ | 37 |
| AAV-IFNβ/α1/γ | 32 |
| co_IFNβ/α1/γ | 38 |

### 8. Definitions & Conventions

As used herein, the term “Inverted Terminal Repeat” (ITR) refers to symmetrical nucleic acid sequences in the genome of adeno-associated viruses required for efficient replication and encapsidation. ITR sequences are located at each end of the AAV DNA genome. The ITRs serve as the origins of replication for viral DNA synthesis and are essential cis elements for generating AAV vectors.

The terms “peptide,” “polypeptide,” and “protein” are used interchangeably herein to refer to a polymer of amino acid residues.

The term “linker-peptide”, as used herein, refers to synthetic amino acid sequences that connect or link two polypeptide sequences, e.g., that link two polypeptide domains. As used herein the term “synthetic” refers to amino acid sequences that are not naturally occurring.

The term “self-cleaving peptide” as used herein refers to a peptide sequence that is associated with a cleavage activity that occurs between two amino acid residues within the peptide sequence itself. For example, in 2A peptides, cleavage occurs between a glycine residue a proline residue. This occurs through a ‘ribosomal skip mechanism’ during translation, wherein normal peptide bond formation between the glycine residue and the proline residue of the 2A peptide is impaired, without affecting the translation of the rest of the 2A peptide.

As used herein the terms “3-prime” and “5-prime” take their usual meanings in the art to distinguish the ends of polynucleotides, i.e. 5′ and a 3′ end.

A “promoter” refers to an untranslated nucleic acid sequence typically upstream of a coding region that contains the binding site for RNA polymerase and initiates transcription of the DNA. The promoter region may also include other elements that act as regulators of gene expression.

The term “enhancer” refers to a nucleic acid sequence that can stimulate promoter activity and may be an innate element of the promoter or a heterologous element inserted to enhance the level or tissue specificity of a promoter. It is capable of functioning even when moved either upstream or downstream from the promoter.

As used herein, the term “transcriptional regulatory elements” refers to DNA sequences, such as initiator sequences, enhancer sequences, and promoter sequences, which induce, repress, or otherwise control the transcription of protein encoding nucleic acid sequences to which they are operably linked.

As used herein, the term “operably linked” refers to a linkage in which the transcriptional regulatory elements are contiguous with a transgene to control expression of the transgene, as well as transcriptional regulatory elements that act in trans or at a distance to control expression of the transgene.

The terms “identical” or percent “sequence identity,” in the context of two or more nucleic acids or polypeptide sequences, refer to two or more sequences or subsequences that are the same (“identical”) or have a specified percentage of amino acid residues or nucleotides that are the same (i.e., at least about 70% identity, at least about 75% identity, at least 80% identity, at least about 90% identity, preferably at least about 91%, 92%, 93%, 94%, 95%, 96%, 97%, 98%, 99%, or higher identity over the entire sequence of a specified region, when compared and aligned for maximum correspondence over a comparison window or designated region. Methods of alignment of sequences for comparison are well-known in the art. Optimal alignment of sequences for comparison can be conducted, e.g., by the local homology algorithm of Smith & Waterman, Adv. Appl. Math. 2:482 (1981). Additional methods include the homology alignment algorithm of Needleman & Wunsch, J. Mol. Biol. 48:443 (1970) and the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (www.ncbi.nlm.nih.gov/).

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

The term “treatment” or any grammatical variation thereof (e.g., treat, treating, etc.), refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, increasing overall survival (OS), increasing progression-free survival (PFS), decreasing the rate of disease progression, amelioration or palliation of the disease state, and remission or improved prognosis.

The term “subject” or “patient” refers to a human or an animal (particularly a mammal) and other organisms that receive either prophylactic or therapeutic treatment. For example, a subject can be a human, a dog, or a mouse.

“Pharmaceutically acceptable excipient” and “pharmaceutically acceptable carrier” are used interchangeably and refer to a substance that aids the administration of an active agent to and absorption by a subject and can be included in the compositions of the present invention without causing a significant adverse toxicological effect on the patient.

As used herein, the term “administering”, “administration”, or “administer” means delivering the pharmaceutical composition as described herein to a target cell or a subject. The pharmaceutical compositions described herein are designed for delivery to subjects in need thereof by any suitable route or a combination of different routes. In particular embodiments, pharmaceutical compositions are administered by intratumoral injection.

As used herein, the term “cancer” refers to all types of cancer, neoplasm or malignant tumors found in mammals, including leukemia, carcinomas and sarcomas.

“Tumor,” as used herein, refers to all neoplastic cell growth and proliferation and cancerous cells and tissues.

The term “glioblastoma” and “glioblastoma multiforme (GBM)” and “grade IV glioma” are used interchangeably and refer to a brain tumor derived from glial cells (glioma) characterized by the presence of small areas of necrotizing tissue that is surrounded by anaplastic cells.

Unless otherwise indicated, nucleotide sequences are presented 5′ to 3′.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

### 9. Summary of Sequences

| SEQ | Description |
|---|---|
| 1 | CMV enhancer nucleic acid sequence |
| 2 | CBA promoter element nucleic acid sequence |
| 3 | Rabbit beta-globin splice acceptor nucleic acid sequence |
| 4 | Spacer nucleic acid sequence |
| 5 | 5′ spacer sequence |
| 6 | CAG promoter nucleic acid sequence |
| 7 | Human IFNβ protein |
| 8 | Human IFNβ nucleic acid sequence |
| 9 | Codon optimized human IFNβ nucleic acid sequence |
| 10 | Human IFNα1 protein |
| 11 | Human IFNα1 nucleic acid sequence |
| 12 | Codon optimized human IFNα1 nucleic acid sequence |
| 13 | Human IFNγ protein |
| 14 | Human IFNγ nucleic acid sequence |
| 15 | Codon optimized human IFNγ nucleic acid sequence |
| 16 | Spacer sequence |
| 17 | P2A peptide |
| 18 | T2A peptide |
| 19 | Mouse IFNβ protein |
| 20 | Mouse IFNβ nucleic acid sequence |
| 21 | Canine IFNβ protein |
| 22 | Canine IFNβ nucleic acid sequence |
| 23 | WPRE nucleic acid sequence |
| 24 | SV40 poly(A) nucleic acid sequence |
| 25 | v1: AAV-CAG-hIFNb-WPRE-SV40pA: vector comprising a CAG promoter sequence and |
| | a transgene comprising a sequence encoding human IFN beta (hIFNβ) |
| 26 | v1: AAV-CAG-mIFNb-WPRE-SV40pA: vector comprising a CAG promoter sequence and |
| | a transgene comprising a sequence encoding mouse IFN beta (mIFNβ) |
| 27 | v1: AAV-CAG-cIFNb-WPRE-SV40pA: vector comprising a CAG promoter sequence and |
| | a transgene comprising a sequence encoding canine IFN beta (cIFNβ) |
| 28 | v2: AAV-CAG-hIFNa1-WPRE-SV40pA: vector comprising a CAG promoter sequence and |
| | a transgene comprising a sequence encoding human IFN alpha 1 (hIFNα1) |
| 29 | v2: AAV-CAG-hIFNg-WPRE-SV40pA: vector comprising a CAG promoter sequence and |
| | a transgene comprising a sequence encoding human IFN gamma (hIFNγ) |
| 30 | v2: AAV-CAG-hIFNb-GSG-P2A-hIFNa-WPRE-SV40pA: vector comprising a CAG |
| | promoter sequence and a transgene comprising a sequence encoding hIFNβ and hIFNα1 |
| 31 | v2: AAV-CAG-hIFNb-GSG-P2A-hIFNg-WPRE-SV40pA: vector comprising a CAG |
| | promoter sequence and a transgene comprising a sequence encoding hIFNβ and hIFNγ |
| 32 | v2: AAV-CAG-hIFNb-GSG-P2A-hIFNa-GSG-T2A-hIFNg-WPRE-SV40pA: vector comprising |
| | a CAG promoter sequence and a transgene comprising a sequence encoding hIFNβ, |
| | hIFNα1, and hIFNγ |
| 33 | v2: AAV-CAG-hIFNb.co-WPRE-SV40pA: vector comprising a CAG promoter sequence |
| | and a transgene comprising a codon-optimized sequence encoding hIFNβ |
| 34 | v2: AAV-CAG-hIFNa1.co-WPRE-SV40pA: vector comprising a CAG promoter sequence |
| | and a transgene comprising a codon-optimized sequence encoding hIFNα1 |
| 35 | v2: AAV-CAG-hIFNg.co-WPRE-SV40pA: vector comprising a CAG promoter sequence |
| | and a transgene comprising a codon-optimized sequence encoding human hIFNγ |
| 36 | v2: AAV-CAG-hIFNb-GSG-P2A-hIFNa.co-WPRE-SV40pA: vector comprising a CAG |
| | promoter sequence and a transgene comprising a codon-optimized sequence encoding |
| | hIFNβ and hIFNα1 |
| 37 | v2: AAV-CAG-hIFNb-GSG-P2A-hIFNg.co-WPRE-SV40pA: vector comprising a CAG |
| | promoter sequence and a transgene comprising a codon-optimized sequence |
| | encoding hIFNβ and hIFNγ |
| 38 | v2: AAV-CAG-hIFNb-GSG-P2A-hIFNa-GSG-T2A-hIFNg.co-WPRE-SV40pA: vector |
| | comprising a CAG promoter sequence and a transgene comprising a codon-optimized |
| | sequence encoding hIFNβ, hIFNα1, and hIFNγ |

### 10. Annotation for SEQ ID NOS: 25-38

AAV-CAG-hIFNb (SEQ ID NO: 25)

| | |
|---|---|
| LTR | 1..128 |
| | /label = AAV2 L ITR |
| promoter | 192..1124 |
| | /label = CAG Promoter |
| misc_feature | 192..437 |
| | /label = CMV early enhancer |
| promoter | 439..716 |
| | /label = chicken beta-actin promoter |
| misc_feature | 1032..1071 |
| | /label = Rabbit B-globin splice acceptor |
| −10_signal | 1137..1142 |
| | /label = Kozak |
| CDS | 1143..1706 |
| | /codon_start = 1 |
| | /label = Human IFNb |

/translation = ″MTNKCLLQIALLLCFSTTALSMSYNLLGFLQRSSNFQCQKLLWQL
NGRLEYCLKDRMNEDI PEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| misc_feature | 1731..2319 |
| | /label = WPRE |
| | /note = ″woodchuck hepatitis virus |
| | posttranscriptional regulatory element″ |
| polyA_signal | 2362..2483 |
| | /label = SV40 pA |
| | /note = ″SV40 polyadenylation signal″ |
| LTR | complement (2645..2795) |
| | /label = AAV2 R ITR |
| | /label = ITR |

AAV-CAG-mIFNb.gb (SEQ ID NO: 26)

| | |
|---|---|
| LTR | 1..128 |
| | /label = L ITR |
| promoter | 192..1124 |
| | /label = CAG Promoter |
| enhancer | 192..437 |
| | /label = CMV early enhancer |
| promoter | 439..716 |
| | /label = chicken beta-actin promoter |
| misc_feature | 1032..1071 |
| | /label = Rabbit B-globin splice acceptor |
| −10_signal | 1137..1142 |
| | /label = Kozak |
| CDS | 1143..1691 |
| | /codon_start = 1 |
| | /label = Mouse IFNb |

/translation = ″MNNRWILHAAFLLCESTTALSINYKQLQLQERTNIRKCQELLEQL
NGKINLTYRADFKIPMEMTEKMQKSYTAFAIQEMLQNVELVERNNFSSTGWNETIVVRL
LDELHQQTVELKTVLEEKQEERLTWEMSSTALHLKSYYWRVQRYLKLMKYNSYAWMVVR
AEIFRNFLIIRRLTRNFQN″

| | |
|---|---|
| regulatory | 1716..2304 |
| | /regulatory_class = ″response_element″ |
| | /label = WPRE |
| | /note = ″woodchuck hepatitis virus post- |
| | transcriptional regulatory element″ |
| polyA_signal | 2347..2468 |
| | /label = SV40 pA |
| | /note = ″SV40 polyadenylation signal″ |
| LTR | complement (2630..2780) |
| | /label = R ITR |
| | /label = ITR |

AAV-CAG-cIFNb (SEQ ID NO: 27)

| | |
|---|---|
| LTR | 1..128 |
| | /label = L ITR |
| promoter | 192..1124 |
| | /label = CAG Promoter |
| misc_feature | 192..437 |
| | /label = CMV early enhancer |
| promoter | 439..716 |
| | /label = chicken beta-actin promoter |
| misc_feature | 1032..1071 |
| | /label = Rabbit B-globin splice acceptor |
| −10_signal | 1137..1142 |
| | /label = Kozak |
| CDS | 1143..1703 |
| | /codon_start = 1 |
| | /label = Canine IFNb |

/translation = ″MTSRCILQTTLLLYFSTMALAMSNDLLRSQLSSSSLECQELLLQL
NGTTEYCLKDRINFEIPEEIEKSRQFQKEDIILITHEMFQKIFDIFRRNISRTGWNETT
VENLLVKLHWQKEHLEIILEDVKEKENFTWDNRTLLHLKKYYLRIVQYLKAKEYSICAW
TIVQAEICRNFFELNILTDYLQN″

| | |
|---|---|
| regulatory | 1728..2316 |
| | /regulatory_class = ″response_element″ |
| | /label = WPRE |
| | /note = ″woodchuck hepatitis virus post- |
| | transcriptional regulatory element″ |
| polyA_signal | 2359..2480 |
| | /label = SV40 pA |
| | /note = ″SV40 polyadenylation signal″ |
| LTR | complement (2642..2792) |
| | /label = AAV2 R ITR |
| | /label = AAV2 ITR |
| | /label = ITR |

AAV-CAG-hIFNa1 (SEQ ID NO: 28)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc_feature | 1037..1076 |
| | /label = Rabbit B-globin splice acceptor |
| −10_signal | 1142..1151 |
| | /label = Kozak |
| | /note = ″vertebrate consensus sequence for strong |
| | initiationof translation (Kozak, 1987) ″ |
| CDS | 1148..1717 |
| | /codon_start = 1 |
| | /label = Human IFN alpha-1 |

/translation = ″MASPFALLMVLVVLSCKSSCSLGCDLPETHSLDNRRTLMLLAQMS
RISPSSCLMDRHDFGFPQEEFDGNQFQKAPAISVLHELIQQIFNLFTTKDSSAAWDEDL
LDKFCTELYQQLNDLEACVMQEERVGETPLMNADSILAVKKYFRRITLYLTEKKYSPCA
WEVVRAEIMRSLSLSTNLQERLRRKE″

| | |
|---|---|
| regulatory | 1742..2330 |
| | /regulatory_class = ″response_element″ |
| | /label = WPRE |
| | /note = ″woodchuck hepatitis virus post- |
| | transcriptionalregulatory element″ |
| polyA_signal | 2373..2494 |
| | /label = SV40 pA |
| | /label = SV40 poly (A) signal |
| | /note = ″SV40 polyadenylation signal″ |
| LTR | complement (2656..2806) |
| | /label = AAV2 R ITR |
| | /label = ITR |

AAV-CAG-hIFNg (SEQ ID NO: 29)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc_feature | 1037..1076 |
| | /label = Rabbit B-globin splice acceptor |
| −10_signal | 1142..1147 |
| | /label = Kozak |
| CDS | 1148..1648 |
| | /codon_start = 1 |
| | /label = Human IFN gamma |

/translation = ″MKYTSYILAFQLCIVLGSLGCYCQDPYVKEAENLKKYFNAGHSDV
ADNGTLFLGILKNWKEESDRKIMQSQIVSFYFKLFKNFKDDQSIQKSVETIKEDMNVKF
FNSNKKKRDDFEKLTNYSVTDLNVQRKAIHELIQVMAELSPAAKTGKRKRSQMLERGRR
ASQ″

| | |
|---|---|
| regulatory | 1673..2261 |
| | /regulatory_class = ″response_element″ |
| | /label = WPRE |
| | /note = ″woodchuck hepatitis virus |
| | post-transcriptionalregulatory element″ |
| polyA_signal | 2304..2425 |
| | /label = SV40 poly (A) signal |
| | /note = ″SV40 polyadenylation signal″ |
| LTR | complement (2587..2737) |
| | /label = AAV2 R ITR |
| | /label = ITR |

AAV-CAG-IFNb-P2A-hIFNa1 (SEQ ID NO: 30)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc feature | 1037..1076 |
| | /label = Rabbit B-globin splice acceptor |
| −10_signal | 1142..1147 |
| | /label = Kozak |
| CDS | 1148..1711 |
| | /codon_start = 1 |
| | /label = Human IFNb |

/translation = ″MTNKCLLQIALLLCFESTTALSMSYNLLGFLQRSSNFQCQKLLWQL
NGRLEYCLKDRMNEDIPEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| misc_feature | 1712..1720 |
| | /label = GSG linker |
| CDS | 1721..1777 |
| | /codon_start = 1 |
| | /product = ″2A peptide from porcine teschovirus- |
| | 1polyprotein″ |
| | /label = P2A |
| | /note = ″Eukaryotic ribosomes fail to insert a |
| | peptide bond between the Gly and Pro residues, |
| | yielding separate polypeptides.″ |
| CDS | /translation = ″ATNFSLLKQAGDVEENPGP″ |
| | 1778..2260 |
| | /codon_start = 1 |
| | /label = Human IFNa |

/translation = ″HSLDNRRTLMLLAQMSRISPSSCLMDRHDFGFPQEEFDGNQFQKA
PAISVLHELIQQIFNLFTTKDSSAAWDEDLLDKFCTELYQQLNDLEACVMQEERVGETP
LMNADSILAVKKYFRRITLYLTEKKYSPCAWEVVRAEIMRSLSLSTNLQERLRRKE″

| | |
|---|---|
| regulatory | 2279..2867 |
| | /regulatory_class = ″response_element″ |
| | /label = WPRE |
| | /note = ″woodchuck hepatitis virus |
| | post-transcriptionalregulatory element″ |
| polyA_signal | 2910..3031 |
| | /label = SV40 poly (A) signal |
| | /note = ″SV40 polyadenylation signal″ |
| LTR | complement (3193..3343) |
| | /label = AAV2 R ITR |
| | /label = ITR |

AAV-CAG-HIFNb-P2A-hIfNg (SEQ ID NO: 31)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc feature | 1037..1076 |
| | /label = Rabbit B-globin splice acceptor |
| −10_signal | 1142..1147 |
| | /label = Kozak |
| CDS | 1148..1711 |
| | /codon_start = 1 |
| | /label = Human IFNb |

/translation = ″MTNKCLLQIALLLCFSTTALSMSYNLLGFLQRSSNFQCQKLLWQL
NGRLEYCLKDRMNFDIPEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| misc_feature | 1712..1720 |
| | /label = GSG linker |
| CDS | 1721..1777 |
| | /codon_start = 1 |
| | /product-″2A peptide from porcine teschovirus- |
| | 1polyprotein″ |
| | /label = P2A |
| | /note = ″Eukaryotic ribosomes fail to insert a |
| | peptide bond between the Gly and Pro residues, |
| | yielding separate polypeptides.″ |
| | /translation = ″ATNFSLLKQAGDVEENPGP″ |
| CDS | 1778..2278 |
| | /codon_start = 1 |
| | /label = Human IFNg |
| | /label = Human IFN gamma |

/translation = ″MKYTSYILAFQLCIVLGSLGCYCQDPYVKEAENLKKYFNAGHSDV
ADNGTLFLGILKNWKEESDRKIMQSQIVSFYFKLFKNFKDDQSIQKSVETIKEDMNVKF
FNSNKKKRDDFEKLTNYSVTDLNVQRKAIHELIQVMAELSPAAKTGKRKRSQMLERGRR
ASQ″

| | |
|---|---|
| regulatory | 2297..2885 |
| | /regulatory_class = ″response_element″ |
| | /label = WPRE |
| | /note = ″woodchuck hepatitis virus |
| | post-transcriptionalregulatory element″ |
| polyA_signal | 2928..3049 |
| | /label = SV40 poly (A) signal |
| | /note = ″SV40 polyadenylation signal″ |
| LTR | complement (3211..3361) |
| | /label = AAV2 R ITR |
| | /label = ITR |

AAV-CAG-hIFNb-P2A-hIFNa1-T2A-hIFNG (SEQ ID NO: 32)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc feature | 1037..1076 |
| | /label = Rabbit B-globin splice acceptor |
| −10_signal | 1142..1147 |
| | /label = Kozak |
| CDS | 1148..1711 |
| | /codon_start = 1 |
| | /label = Human IFNb |

/translation = ″MTNKCLLQIALLLCFSTTALSMSYNLLGFLQRSSNFQCQKLLWQL
NGRLEYCLKDRMNEDIPEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| misc_feature | 1712..1720 |
| | /label = GSG linker |
| CDS | 1721..1777 |
| | /codon_start = 1 |
| | /product = ″2A peptide from porcine teschovirus- |
| | 1polyprotein″ |
| | /label = P2A |
| | /note = ″Eukaryotic ribosomes fail to insert a |
| | peptide bond between the Gly and Pro residues, |
| | yielding separate polypeptides.″ |
| | /translation = ″ATNFSLLKQAGDVEENPGP″ |
| CDS | 1778..2260 |
| | /codon_start = 1 |
| | /label = Human IFNa |

/translation = ″HSLDNRRTLMLLAQMSRISPSSCLMDRHDFGFPQEEFDGNQFQKA
PAISVLHELIQQIFNLFTTKDSSAAWDEDLLDKFCTELYQQLNDLEACVMQEERVGETP
LMNADSILAVKKYFRRITLYLTEKKYSPCAWEVVRAEIMRSLSLSTNLQERLRRKE″

| | |
|---|---|
| misc_feature | 2261..2269 |
| | /label = GSG linker |
| CDS | 2270..2323 |
| | /codon_start = 1 |
| | /product = ″2A peptide from Thosea asigna |
| | viruscapsidprotein″ |
| | /label = T2A |
| | /note = ″Eukaryotic ribosomes fail to insert a |
| | peptide bond |
| | between the Gly and Pro residues, yielding |
| | separate polypeptides.″ |
| | /translation = ″EGRGSLLTCGDVEENPGP″ |
| CDS | 2324..2824 |
| | /codon_start = 1 |
| | /label = Human IFNg |
| | /label = Human IFN gamma |

/translation = ″MKYTSYILAFQLCIVLGSLGCYCQDPYVKEAENLKKYFNAGHSDV
ADNGTLFLGILKNWKEESDRKIMQSQIVSFYFKLFKNFKDDQSIQKSVETIKEDMNVKF
FNSNKKKRDDFEKLTNYSVTDLNVQRKAIHELIQVMAELSPAAKTGKRKRSQMLERGRR
ASQ″

| | |
|---|---|
| regulatory | 2843..3431 |
| | /regulatory_class = ″response_element″ |
| | /label = WPRE |
| | /note = ″woodchuck hepatitis virus |
| | post-transcriptionalregulatory element″ |
| polyA_signal | 3474..3595 |
| | /label = SV40 poly (A) signal |
| | /note = ″SV40 polyadenylation signal″ |
| LTR | complement (3757..3907) |
| | /label = AAV2 R ITR |
| | /label = ITR |

AAV-CAG-hIFNb-Codon Optimized (SEQ ID NO: 33)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443.720 |
| | /label = chicken beta-actin promoter |
| misc_feature | 1037..1076 |
| | /label = Rabbit B-globin slice acceptor |
| −10_signal | 1142..1147 |
| | /label = Kozak |
| CDS | 1148..1711 |
| | /codon_start = 1 |
| | /label = hIFNb_CO |

/translation = ″MTNKCLLQIALLLCFSTTALSMSYNLLGFLQRSSNFQCQKLLWQL
NGRLEYCLKDRMNEDIPEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| regulatory | 1736..2324 |
| | /regulatory_class = ″response_element″ |
| | /label = WPRE |
| | /note = ″woodchuck hepatitis virus |
| | post-transcriptionalregulatory element″ |
| polyA_signal | 2367..2488 |
| | /label = SV40 poly (A) signal |
| | /note = ″SV40 polyadenylation signal″ |
| ltr | complement (2650..2800) |
| | /label = AAV2 R ITR |
| | /label = ITR |

AAV-CAG-hIFNa1-Codon Optimized (SEQ ID NO: 34)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc feature | 1037..1076 |
| | /label = Rabbit B-globin splice acceptor |
| −10_signal | 1142..1151 |
| | /label = Kozak |
| | /note = ″vertebrate consensus sequence for strong |
| | initiationof translation (Kozak, 1987) ″ |
| CDS | 1148..1717 |
| | /codon_start = 1 |
| | /label = hIFNa1_CO |

/translation = ″MASPFALLMVLVVLSCKSSCSLGCDLPETHSLDNRRTLMLLAQMS
RISPSSCLMDRHDFGFPQEEFDGNQFQKAPAISVLHELIQQIFNLFTTKDSSAAWDEDL
LDKFCTELYQQLNDLEACVMQEERVGETPLMNADSILAVKKYFRRITLYLTEKKYSPCA
WEVVRAEIMRSLSLSTNLQERLRRKE″

| | |
|---|---|
| misc_feature | 1742..2330 |
| | /label = WPRE |
| | /note = ″woodchuck hepatitis virus |
| | post-transcriptionalregulatory element″ |
| polyA_signal | 2373..2494 |
| | /label = SV40 poly (A) signal |
| | /note = ″SV40 polyadenylation signal″ |
| LTR | complement (2656..2806) |
| | /label = AAV2 R ITR |
| | /label = ITR |

AAV-CAG-hIFNG Codon Optimized (SEQ ID NO: 35)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc_feature | 1037..1076 |
| | /label = Rabbit B-globin splice acceptor |
| −10_signal | 1142..1147 |
| | /label = Kozak |
| CDS | 1148..1648 |
| | /codon_start = 1 |
| | /label = hIFNg_CO |

/translation = ″MKYTSYILAFQLCIVLGSLGCYCQDPYVKEAENLKKYFNAGHSDV
ADNGTLFLGILKNWKEESDRKIMQSQIVSFYFKLFKNEKDDQSIQKSVETIKEDMNVKF
FNSNKKKRDDFEKLTNYSVTDLNVQRKAIHELIQVMAELSPAAKTGKRKRSQMLERGRR
ASQ″

| | |
|---|---|
| regulatory | 1673..2261 |
| | /regulatory_class = ″response_element″ |
| | /label = WPRE |
| | /note = ″woodchuck hepatitis virus |
| | post-transcriptionalregulatory element″ |
| polyA_signal | 2304..2425 |
| | /label = SV40 poly (A) signal |
| | /note = ″SV40 polyadenylation signal″ |
| LTR | complement (2587..2737) |
| | /label = AAV2 R ITR |
| | /label = ITR |

AAV-CAG-hIFNb-P2A-hIFNa1 Codon Optimized (SEQ ID NO: 36)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc_feature | 1037..1076 |
| | /label = Rabbit B-globin splice acceptor |
| −10_signal | 1142..1147 |
| | /label = Kozak |
| CDS | 1148..1711 |
| | /codon_start = 1 |
| | /label = hIFNb_CO |

/translation = ″MTNKCLLQIALLLCFSTTALSMSYNLLGFLQRSSNFQCQKLLWQL
NGRLEYCLKDRMNFDIPEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| misc_feature | 1712..1720 |
| | /label = GSG linker |
| CDS | 1721..1777 |
| | /codon_start = 1 |
| | /product = ″2A peptide from porcine teschovirus- |
| | 1polyprotein″ |
| | /label = P2A |
| | /note = ″Eukaryotic ribosomes fail to insert a |
| | peptide bond |
| | between the Gly and Pro residues, yielding |
| | separate polypeptides.″ |
| | /translation = ″ATNFSLLKQAGDVEENPGP″ |
| CDS | 1778..2347 |
| | /codon_start = 1 |
| | /label = hIFNa1_CO |

/translation = ″MASPFALLMVLVVLSCKSSCSLGCDLPETHSLDNRRTLMLLAQMS
RISPSSCLMDRHDFGFPQEEFDGNQFQKAPAISVLHELIQQIFNLFTTKDSSAAWDEDL
LDKFCTELYQQLNDLEACVMQEERVGETPLMNADSILAVKKYFRRITLYLTEKKYSPCA
WEVVRAEIMRSLSLSTNLQERLRRKE″

| | |
|---|---|
| regulatory | 2366..2954 |
| | /regulatory_class = ″ribosome_binding_site″ |
| | /label = WPRE |
| | /note = ″woodchuck hepatitis |
| | virusposttranscriptionalregulatory element″ |
| polyA_signal | 2997..3118 |
| | /label = SV40 poly (A) signal |
| | /note = ″SV40 polyadenylation signal″ |
| LTR | complement (3280..3430) |
| | /label = AAV2 R ITR |
| | /label = ITR |

AAV-CAG-hIFNb-P2A-hIFNg Codon Optimized (SEQ ID NO: 37)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc_feature | 1037..1076 |
| | /label = Rabbit B-globin splice acceptor |
| −10_signal | 1142..1147 |
| | /label = Kozak |
| CDS | 1148..1711 |
| | /codon_start = 1 |
| | /label = hIFNb_CO |

/translation = ″MTNKCLLQIALLLCFESTTALSMSYNLLGFLQRSSNFQCQKLLWQL
NGRLEYCLKDRMNEDIPEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| misc_feature | 1712..1720 |
| | /label = GSG linker |
| CDS | 1721..1777 |
| | /codon_start = 1 |
| | /product = ″2A peptide from porcine teschovirus- |
| | 1polyprotein″ |
| | /label = P2A |
| | /note = ″Eukaryotic ribosomes fail to insert a |
| | peptide bond |
| | between the Gly and Pro residues, yielding |
| | separate polypeptides.″ |
| | /translation = ″ATNFSLLKQAGDVEENPGP″ |
| CDS | 1778..2278 |
| | /codon_start = 1 |
| | /label = hIFNg_CO |

/translation = ″MKYTSYILAFQLCIVLGSLGCYCQDPYVKEAENLKKYFNAGHSDV
ADNGTLFLGILKNWKEESDRKIMQSQIVSFYFKLFKNFKDDQSIQKSVETIKEDMNVKF
FNSNKKKRDDFEKLTNYSVTDLNVQRKAIHELIQVMAELSPAAKTGKRKRSQMLERGRR
ASQ″

| | |
|---|---|
| regulatory | 2297..2885 |
| | /regulatory_class = ″response_element″ |
| | /label = WPRE |
| | /note = ″woodchuck hepatitis virus |
| | post-transcriptionalregulatory element″ |
| polyA_signal | 2928..3049 |
| | /label = SV40 poly (A) signal |
| | /note = ″SV40 polyadenylation signal″ |
| LTR | complement (3211..3361) |
| | /label = AAV2 R ITR |
| | /label = ITR |

AAV-CAG-hIFNb-P2A-hIFNa1-T2A-hIFNg Codon Optimized (SEQ ID NO: 38)

| | |
|---|---|
| LTR | 1..110 |
| | /label = AAV2 L ITR |
| | /label = ITR |
| promoter | 196..1129 |
| | /label = CAG Promoter |
| enhancer | 196..441 |
| | /label = CMV early enhancer |
| promoter | 443..720 |
| | /label = chicken beta-actin promoter |
| misc feature | 1037..1076 |
| | /label = Rabbit B-globin splice acceptor |
| −10_signal | 1142..1147 |
| | /label = Kozak |
| CDS | 1148..1711 |
| | /codon_start = 1 |
| | /label = hIFNb_CO |

/translation = ″MTNKCLLQIALLLCFSTTALSMSYNLLGFLQRSSNFQCQKLLWQL
NGRLEYCLKDRMNFDIPEEIKQLQQFQKEDAALTIYEMLQNIFAIFRQDSSSTGWNETI
VENLLANVYHQINHLKTVLEEKLEKEDFTRGKLMSSLHLKRYYGRILHYLKAKEYSHCA
WTIVRVEILRNFYFINRLTGYLRN″

| | |
|---|---|
| misc_feature | 1712..1720 |
| | /label = GSG linker |
| CDS | 1721..1777 |
| | /codon_start = 1 |
| | /product = ″2A peptide from porcine teschovirus- |
| | 1polyprotein″ |
| | /label = P2A |
| | /note = ″Eukaryotic ribosomes fail to insert a |
| | peptide bond |
| | between the Gly and Pro residues, yielding |
| | separate polypeptides.″ |
| | /translation = ″ATNFSLLKQAGDVEENPGP″ |
| CDS | 1778..2347 |
| | /codon_start = 1 |
| | /label = hIFNa1_CO |

/translation = ″MASPFALLMVLVVLSCKSSCSLGCDLPETHSLDNRRTLMLLAQMS
RISPSSCLMDRHDFGFPQEEFDGNQFQKAPAISVLHELIQQIFNLFTTKDSSAAWDEDL
LDKFCTELYQQLNDLEACVMQEERVGETPLMNADSILAVKKYFRRITLYLTEKKYSPCA
WEVVRAEIMRSLSLSTNLQERLRRKE″

| | |
|---|---|
| misc_feature | 2348..2356 |
| | /label = GSG linker |
| CDS | 2357..2410 |
| | /codon_start = 1 |
| | /product = ″2A peptide from Thosea asigna |
| | viruscapsidprotein″ |
| | /label = T2A |
| | /note = ″Eukaryotic ribosomes fail to insert a |
| | peptide bond between the Gly and Pro residues, |
| | yielding separate polypeptides.″ |
| | /translation = ″EGRGSLLTCGDVEENPGP″ |
| CDS | 2411..2911 |
| | /codon_start = 1 |
| | /label = hIFNg_CO |

/translation = ″MKYTSYILAFQLCIVLGSLGCYCQDPYVKEAENLKKYFNAGHSDV
ADNGTLFLGILKNWKEESDRKIMQSQIVSFYFKLFKNFKDDQSIQKSVETIKEDMNVKE
FNSNKKKRDDFEKLTNYSVTDLNVQRKAIHELIQVMAELSPAAKTGKRKRSQMLERGRR
ASQ″

| | |
|---|---|
| regulatory | 2930..3518 |
| | /regulatory_class = ″response_element″ |
| | /label = WPRE |
| | /note = ″woodchuck hepatitis virus |
| | post-transcriptionalregulatory element″ |
| polyA_signal | 3561..3682 |
| | /label = SV40 poly (A) signal |
| | /note = ″SV40 polyadenylation signal″ |
| LTR | complement (3844..3994) |
| | /label = AAV2 R ITR |
| | /label = ITR |

***

All publications and patent documents cited herein are incorporated herein by reference as if each such publication or document was specifically and individually indicated to be incorporated herein by reference. Citation of publications and patent documents (patents, published patent applications, and unpublished patent applications) is not intended as an admission that any such document is pertinent prior art, nor does it constitute any admission as to the contents or date of the same.

What is claimed is:

1. A method for treating a patient in need of treatment for glioblastoma, comprising administering an interferon (IFN) alpha, IFN beta, IFN gamma, a combination of any two of IFN-alpha, IFN-beta, and IFN gamma, or all three of IFN alpha, IFN beta and IFN gamma, wherein the administering comprises gene therapy with a single viral vector.

2. The method of claim 1 wherein a combination of any two of IFN-alpha, IFN-beta, and IFN gamma, or all three of IFN alpha, IFN beta or IFN gamma are encoded in a polycistronic transgene and an AAV viral vector comprising the transgene is administered.

3. The method of claim 1 or 2 wherein interferon beta is administered.

4. The method of and of claims 1 to 3 wherein the viral vector is AAV and comprises adeno-associated virus 9 capsid.

5. The any of claims 1 to 4 wherein the viral vector is administered by Convection Enhanced Delivery (CED).

6. A recombinant adeno-associated virus (AAV) vector comprising an expression cassette comprising
a) a CAG promoter,
wherein the CAG promoter comprises
(i) a first segment comprising a cytomegalovirus (CMV) enhancer sequence,
(ii) a second segment comprising a chicken beta-actin (CBA) gene promoter element,
(iii) a third segment comprising a spacer sequence, and
(iv) a fourth segment comprising a rabbit beta-globin splice acceptor,
wherein the order of the segments 5-prime to 3-prime is first, second, third, and fourth, and
b) a transgene comprising a sequence encoding a first interferon polypeptide, wherein the transgene is 3-prime to the CAG promoter, and wherein expression of the transgene is under the control of the CAG promoter.

7. The AAV vector of claim 6 wherein the third segment is 250 nucleotides to 350 nucleotides in length measured from the 3-prime end of the CBA promoter and the 5′ end of the rabbit beta-globin splice acceptor.

8. The AAV vector of claim 6 wherein
i) the first segment has the sequence of SEQ ID NO: 1, and/or
ii) the second segment has the sequence of SEQ ID NO: 2, and/or
iii) the third segment has the sequence of SEQ ID NO: 4, and/or
iv) the fourth segment has the sequence of SEQ ID NO: 3.

9. The AAV vector of claim 6 wherein the expression cassette does not comprise SEQ ID NO: 5.

10. The AAV vector of claim 6 wherein the CAG promoter has the sequence of SEQ ID NO: 6.

11. The AAV vector of any of claims 6-9 wherein the first interferon polypeptide is human interferon beta (hIFNβ).

12. The AAV vector of claim 11 wherein the transgene comprises a sequence encoding a second interferon polypeptide, wherein expression of the second interferon polypeptide is under control of the CAG promoter, wherein the sequence encoding the second interferon polypeptide is 3-prime from the sequence encoding the first interferon polypeptide, and wherein the second interferon polypeptide is human interferon alpha 1 (hIFNα1).

13. The AAV vector of claim 11 wherein the sequence encoding the first interferon polypeptide is codon optimized for expression in human cells, or the AAV vector of claim 7 wherein the sequences encoding the first and second interferon polypeptides are codon optimized for expression in human cells.

14. The AAV vector of claim 12 wherein the transgene comprises a sequence encoding a third interferon polypeptide, wherein expression of the third interferon polypeptide is under control of the CAG promoter, wherein the sequence encoding the third interferon polypeptide is 3-prime from the sequence encoding the second interferon polypeptide, and wherein the third interferon polypeptide is human interferon gamma.

15. The AAV vector of claim 14 wherein the sequences encoding the first, second and third interferon polypeptides are codon optimized for expression in human cells.

16. The AAV vector of claim 10 wherein the transgene comprises a sequence encoding a second interferon polypeptide, wherein expression of the second interferon polypeptide is under control of the CAG promoter, wherein the sequence encoding the second interferon polypeptide is 3-prime from the sequence encoding the first interferon polypeptide, and wherein the second interferon polypeptide is human interferon gamma (hIFNγ).

17. The AAV vector of claim 16 wherein the sequences encoding the first and second interferon polypeptides are codon optimized for expression in human cells.

18. The AAV vector of any of claims 6-9 wherein the first interferon polypeptide is hlFNα1.

19. The AAV vector of claim 18 wherein the sequence encoding the first interferon polypeptide is codon optimized for expression in human cells.

20. The AAV vector of any of claims 6-9 wherein the first interferon polypeptide is hIFNγ.

21. The AAV vector of claim 20 wherein the sequence encoding the first interferon polypeptide is codon optimized for expression in human cells.

22. The AAV vector of any of claims 6-9 wherein the first interferon polypeptide is mouse interferon beta (mIFNβ).

23. The AAV vector of any of claims 6-9 wherein the first interferon polypeptide is canine interferon beta (cIFNβ).

24. The AAV vector of any one of claims 12-13 and 16-17, wherein the sequence encoding the first interferon polypeptide and the sequence encoding the second interferon polypeptide are connected by a sequence encoding a first linker peptide and a sequence encoding a first self-cleaving peptide; and wherein the sequence encoding a first self-cleaving peptide is 3-prime from the sequence encoding a first linker peptide.

25. The AAV vector of claim 14 or 15,
wherein the sequence encoding the first interferon polypeptide and the sequence encoding the second interferon polypeptide are connected by a sequence encoding a first linker peptide and a sequence encoding a first self-cleaving peptide,
wherein the sequence encoding a first self-cleaving peptide is 3-prime from the sequence encoding a first linker peptide,
wherein the sequence encoding the second interferon polypeptide and the sequence encoding the third interferon polypeptide are connected by a sequence encoding a second linker peptide and a sequence encoding a second self-cleaving peptide, and
wherein the sequence encoding a second self-cleaving peptide is 3-prime from the sequence encoding a second linker peptide.

26. The AAV vector of any one of claims 6-25, wherein the expression cassette further comprises a woodchuck hepatitis virus posttranscriptional regulatory element (WPRE).

27. The AAV vector of claim 26, wherein the transgene(s) is located between the CAG promoter and the WPRE.

28. The AAV vector of any one of claims 6-27, wherein the expression cassette further comprises a polyadenylation signal.

29. The AAV vector of any one of claims 6-28, wherein the expression cassette comprises two adeno-associated virus (AAV) inverted terminal repeats (ITRs), wherein the CAG promoter and the transgene(s) are located between the two ITRs.

30. The AAV vector of claim 29, wherein the ITR is AAV1 ITR, AAV2 ITR, AAV3 ITR, AAV4 ITR, AAV5 ITR, AAV6 ITR, AAV7 ITR, AAV8 ITR, AAV-rh8 ITR, AAV9 ITR, AAV10 ITR, AAV-rh10 ITR, AAV11 ITR, or AAV12 ITR.

31. The AAV vector of any one of claims 6-30, wherein the expression cassette does not comprise an enhancer sequence other than the CMV enhancer sequence.

32. A recombinant adeno-associated virus (AAV) comprising an AAV capsid and the AAV vector of any one of claims 6-31.

33. The AAV of claim 32 wherein the AAV capsid is AAV1, AAV2, AAV3, AAV3b, AAV4, AAV5, AAV6, AAV7, AAV8, AAV-rh8, AAV9, AAV9-hu14, AAV10, AAV-rh10, AAV11, AAV12, AAV-NP22, AAV-NP66, AAV-NP40, AAV-NP59, AAV-DJ, AAV-DJ/8, AAV-LK03, AAV-rh74, or AAV-hu37.

34. An isolated cell comprising the AAV vector of any one of claims 6-31 or the AAV of claim 32 or claim 33.

35. A pharmaceutical composition comprising the AAV vector of any one of claims 6-31, the AAV of claim 32 or claim 33, or the isolated cell of claim 34, and a pharmaceutically acceptable excipient.

36. A method for treating cancer in a mammal in need of treatment comprising administering the AAV vector of any one of claims 6-31, the AAV of claim 32 or claim 233, the isolated cell of claim 34, or the pharmaceutical composition of claim 30.

37. The method of claim 36, wherein the cancer is glioblastoma.

38. The method of claim 36 wherein the subject is a human, a mouse, or a dog.

39. The method of any of claims 36-38, wherein the method comprises administering the AAV vector, and the AAV vector is administered by intratumoral injection by Convection Enhanced Delivery (CED).

40. The method of any of claim 39, wherein the method comprises administering the AAV vector, and the AAV vector is administered by intratumoral injection, intracranial injection, intracerebral injection, intracerebroventricular, or injection into the Cerebrospinal fluid (CSF) via the cerebral ventricular system, cisterna magna, or intrathecal space.

41. The use of the AAV vector of any one of claims 16-31, the AAV of claim 32 or claim 33, the isolated cell of claim 34, or the pharmaceutical composition of claim 30 for the preparation of a medicament for treating cancer.

42. The AAV vector of any one of claims 6-31, the AAV of claim 32 or claim 33, the isolated cell of claim 34, or the pharmaceutical composition of claim 35 for the preparation of a medicament for treating cancer.
